(19) United States
(12) Patent Application Publication
(10) Pub. No.: US 2022/0056498 A1
(43) Pub. Date: Feb. 24, 2022

(54) COMPOSITIONS AND METHOD FOR SYNTHESIZING NUCLEIC ACIDS

(71) Applicant: PRESIDENT AND FELLOWS OF HARVARD COLLEGE, Cambridge, MA (US)

(72) Inventors: Jocelyn KISHI, Boston, MA (US); Ninning LIU, Boston, MA (US); Sinem SAKA, Allston, MA (US); Peng YIN, Brookline, MA (US)

(73) Assignee: PRESIDENT AND FELLOWS OF HARVARD COLLEGE, Cambridge, MA (US)

(21) Appl. No.: 17/415,326

(22) PCT Filed: Jan. 24, 2020
(86) PCT No.: PCT/US2020/014952

§ 371 Date: Jun. 17, 2021

Related U.S. Application Data
(60) Provisional application No. 62/796,982, filed on Jan. 25, 2019.
(60) Provisional application No. 62/845,539, filed on May 9, 2019.

(51) Int. Cl. C12P 19/34; C07H 21/00

(52) U.S. Cl. CPC C12P 19/34; C07H 21/00

(57) ABSTRACT

Provided herein are compositions, kits and methods for synthesis of nucleic acids. Also provided herein are compositions and methods for synthesizing strands of nucleic acid across different nucleic acid back-bones hybridized together using a strand displacing polymerase.

### CROSS-REFERENCE TO RELATED APPLICATIONS

This application claims benefit under 35 U.S.C. § 119(e) of U.S. Provisional Application No. 62/796,982, filed Jan. 25, 2019, and U.S. Provisional Application No. 62/845,539, filed May 9, 2019, the contents of both of which are incorporated herein by reference in their entireties.

### GOVERNMENT SUPPORT

This invention was made with government support under 1317291 and 1729397 awarded by the National Science Foundation and GM133052 awarded by the National Institute of Health, and under N00014-16-1-2410 and N00014-18-1-2549 awarded by the Department of Defense Office of Naval Research. The government has certain rights in the invention.

### TECHNICAL FIELD

The present disclosure relates to compositions and methods for synthesis of nucleic acids using strand displacing polymerases.

### BACKGROUND

The ability to synthesize arbitrary sequences of nucleic acids, in particular DNA, has revolutionized how scientists can study and engineer biology. Whole genomes can now be sequenced effectively, but the technology to synthesize genomes has lagged behind. Synthetic single-stranded sequences (‘oligos’) are typically synthesized chemically through cyclic coupling steps. However, oligonucleotides are typically not chemically synthesized past two hundred bases, due to the limitations on chemical coupling efficiency. Therefore, synthesis methods have been developed to assemble larger fragments of single-stranded and double-stranded sequences, including enzymatic assembly using sequential or simultaneous combination of multiple enzyme activities (such as restriction enzyme digestion and ligation, or isothermal Gibson assembly which combines 5′ exonuclease, the 3′ extension activity of a DNA polymerase and DNA ligase activity), non-enzymatic twin primer assembly, and chemical assembly (such as click DNA assembly. While these can be effective at producing longer sequences of single-stranded and double-stranded DNA, sequences of hundreds of thousands of bases must be further assembled from shorter fragments in vivo using yeast vectors. A new simple and robust synthesis method capable of generating long sequences of DNA could have important enabling applications in molecular biology, genome engineering, nanotechnology, and polymer-based data storage.

Thus, there remains a need in the art for compositions and methods for synthesizing nucleic acids of arbitrary length prescribed sequences, polymeric sequences of prescribed length, or sequences comprising prescribed number of repeats. The present disclosure addresses some of these needs.

### SUMMARY

Provided herein are compositions and methods for synthesizing strands of nucleic acid across different nucleic acid backbones hybridized together using a strand displacing polymerase. The compositions and methods described herein for synthesizing strands across nucleic acid junctions can effectively decouple the assembly step of nucleic acid template from the synthesis step. This provides, among other things, the ability to sequentially perform robust annealing of guide strands, that can hybridize in predictable ways close to thermodynamic optimum, and follow up with polymerization to create the new transcript. The compositions and methods described herein can allow scaling to very long and arbitrary sequences. Moreover, the entire assembly and synthesis process can happen isothermally, including at room temperature, which enable a generalized workflow for synthesis of long arbitrary sequences under relatively mild conditions.

The methods and compositions provided herein are based, in part, on the discovery that DNA polymerase displacement can be leveraged to synthesize any desired DNA sequence by using a specific nucleic acid template secondary structure (FIG. 1A). Generally, and without limitation, the nucleic acid template can comprise oligonucleotides hybridized together to form a junction or several junctions that stop the DNA polymerase from synthesizing a nucleic acid sequence straight across the guide strand with complementary nucleotides, but allows for the DNA polymerase to displace and cross over the junction. Polymerization can then continue on a second guide strand and copy along a new backbone.

By hybridizing the multiple oligonucleotides together, called guide strands, the cross junction synthesis can be cascaded to form longer sequences (FIG. 2). Multiple guide strands can be hybridized to make up a nucleic acid template of any length. The nucleic acid template can be used for synthesizing a nucleic acid sequence of any desired length.

Accordingly, in one aspect provided herein is a nucleic acid template. For example, a nucleic acid template for use in the synthesis of a nucleic acid sequence by a method described herein. Generally, the nucleic acid template comprises a region, e.g., a double-stranded region, that can inhibit progression of a strand displacing polymerase. The double-stranded region along with the sequence at the 3′-end and the 5′-end can also be referred to as a synthesis domain or synthesis region herein.

As shown in FIG. 1B, an exemplary cross-junction (100) comprises in 3′ to 5′ direction a synthesis region (101), a double-stranded region (102), and another synthesis region (103). The 3′-end (104) of the double-stranded region (102) has a sequence substantially similar to a nucleic acid sequence of the synthesis region (103) at the 5′-end of the duplex. The double-stranded region (102) further comprises a blocking domain (105) at the 5′-end of the region (104), wherein the blocking domain (105) is capable of blocking strand-displacement activity of a polymerase. The cross-junction can be one continuous polynucleotide or the cross-junction can be prepared by annealing two separate guide-strands (106 and 107).

The nucleic acid template can comprise any number of cross-junctions. For example, the nucleic acid template can comprise two or more, e.g., three, four, five, six, seven, eight, nine, ten or more cross-junctions. When the nucleic acid template comprises two or more cross-junctions, they can be linked together via a nucleic acid linker. For example, a 5′-end of a first cross-junction can be linked to a 3′-end of a second cross-junction. Without limitations the linker can be of any desired length and/or nucleotide sequence. For example, the linker can simply be a nucleic acid backbone linkage e.g., phosphodiester linkage. In addition, the nucleic acid linkers can all be the same, all different, or some are the same and some are different.

Furthermore, when the nucleic acid template comprises two or more of the cross-junctions, the two or more of the cross-junctions can all be same, some same, some different, and/or all different. For example, the synthesis region (101) of two or more cross-junction can have a substantially identical nucleotide sequence; the synthesis region (103) of two or more cross junctions can have a substantially identical nucleotide sequence; and/or the synthesis region (101) of a first cross junction can have a substantially identical nucleotide sequence to the synthesis region (103) of a second cross-junction.

A cross-junction can be prepared from two separate nucleic acid stands, e.g., guide strands. Accordingly, in some embodiments, the nucleic acid template comprises a first guide strand comprising in 3′ to 5′ direction a first synthesis region, a second synthesis region, a first junction domain, and a first blocking region; and a second guide strand comprising in 3′ to 5′ direction a second blocking region, a second junction domain and a third synthesis region. The first junction domain comprises a nucleotide sequence substantially identical to a nucleotide sequence of the third synthesis region, and the first junction domain and second junction domain are substantially complementary to each other and form a double-stranded region. The first blocking region and the second blocking region together form a first blocking domain that blocks strand displacement activity of a polymerase. Without limitation, the above-noted regions and domains can independently comprise any nucleotide sequence provided that the nucleotide sequence of the first junction domain of the first guide strand and the third synthesis region of the second guide strand are substantially identical, and the nucleotide sequence of the first junction domain of the first guide strand and second junction domain of the second guide strand are substantially complementary to each other and form a double-stranded region. Without limitations, the nucleic acid template can comprise any number of guide strands.

In another aspect, provided herein is a method for forming a nucleic acid template. The method comprises annealing a first guide strand and a second guide strand. The first guide strand comprises in 3′ to 5′ direction a first synthesis region, a second synthesis region, a first junction domain, and a first blocking region. The second guide strand comprises in 3′ to 5′ direction a second blocking region, a second junction domain and a third synthesis region, wherein the first junction domain comprises a nucleotide sequence substantially identical to a nucleotide sequence of the third synthesis region, and the first junction domain and second junction domain are substantially complementary to each other and form a double-stranded region. The first blocking region and the second blocking region together form a blocking domain that blocks strand displacement activity of a polymerase.

In another aspect, provided herein is a reaction mixture comprising a nucleic acid template provided herein.

In still another aspect, provided herein is a reaction mixture that can be utilized to create the guide strands by addition of junction regions onto existing nucleic acid strands enzymatically or chemically. For example, a reaction mixture that can be utilized to create the guide strands by addition of blocking regions onto existing nucleic acid strands enzymatically or chemically.

In another aspect, provided herein is a kit comprising the nucleic acid template provided herein.

In yet another aspect, provided herein is a kit comprising components or reaction mixture for creating the nucleic acid template described herein from nucleic acid sequences (partial guide strands) by addition of junction regions and/or blocking regions onto existing nucleic acid strands enzymatically or chemically.

Also provided herein is a method for synthesizing a nucleic acid sequence. Generally, the method comprises extending a nucleotide sequence from 3′-end of a primer annealed to the nucleic acid template using a DNA polymerase having strand displacement activity. If a primer is not already annealed to the nucleic acid template, the method can comprise a step of annealing or hybridizing a primer to the nucleic acid template.

In some embodiments, the method for synthesizing a nucleic acid sequence comprises extending a nucleotide sequence from 3′-end of a primer annealed to a guide strand using a DNA polymerase having strand displacement activity and consecutively adding additional guide strands to synthesis reaction, where the nucleotide sequence is extended prior to adding each additional guide strand.

In some embodiments, provided herein is the use of a nucleic acid template for creating combinatorial barcodes to assign unique identities to a target population, wherein the target population is surface positions/partitions, matrices, biomolecules, molecular libraries or biological material (such as vesicles, cells, tissues, organoids, droplets, liposomes, small molecules, beads), and optionally the use comprising splitting the target population prior to addition of a guide strand and pooling the split target population after addition of the guide strand.

### BRIEF DESCRIPTION OF THE DRAWINGS

FIG. 1A is a schematic representation of an exemplary cross junction synthesis mechanism. First, a strand displacing polymerase copies along a template strand until reaching a stopper (step 1). The first template strand (left) is designed to have an x* domain, as shown, so that the complementary x sequence is appended on the 3′ end of the growing strand. A second template strand (right) paired to the first template strand is designed to have an exposed x* binding domain and an x domain bound to the x* domain on the first template strand. When the x domain on the second template strand displaces the newly synthesized x domain on the 3′ end of the growing strand through branch migration (Lee et al., 1970), that strand is then free to pair this domain with the complementary exposed x* domain on the second template strand. At this point, the strand has now successfully ‘hopped’ the junction to hybridize with the backbone of a new strand, and synthesis may continue.

FIG. 1B is a schematic representation of an exemplary cross-junction.

FIG. 2 is schematic representation of an exemplary cascading cross junction synthesis. A multitude of template strands may be hybridized to enable cascaded cross-junction synthesis reactions. Template strands with corresponding motif domains can be hybridized together, and a strand displacing polymerase can be used to copy across each junction. In the example above, a primer with domain a copies bases ending in domain b until it reaches the first stopper, where it can be then displaced and reach across the junction to the b* on the second template strand (from the left). Sequences up to c, d, and e are copied similarly (with optional additional bases in the grey domains intercalated). This method is able to concatenate arbitrary prescribed sequences, and only requires the fundamental domain constraints depicted in FIG. 1.

FIG. 3 is a schematic representation showing synthesis of a fixed number of repeated sequences. By programming unique hybridization domains (1, 2, 3, and 4) for each of the template strands, a fixed-length template concatemer can be formed for cross-junction synthesis. However, each of these template strands can be used for appending the same sequence as a previous junction (e.g. the x y sequence) if desired. This allows a fixed number of repeated sequence domains to be synthesized. For example, as shown in FIG. 3, the sequence x y is copied into a sequence exactly 4 times.

FIGS. 4A-4C are schematic overview of light-directed cross junction (blocking domain) assembly. FIG. 4A shows interstrand crosslinking reaction between complementary sequences can be achieved with a crosslinking base modification (filled cyan circle). (FIG. 4B shows iterative assembly of cross-junctions: (1) An initial docking strand with a crosslinking base modification and hybridization domain 1 is hybridized to a strand with the complement site 1*, the first barcode sequence b1, the next hybridization domain 2 and a crosslinker base. (2) A UV crosslinking light source is used to covalently link the two sequences together, un-crosslinked sequences are washed away and the next iteration of strands carrying the b2 sequence are introduced. (3) UV crosslinking and continuation of a growing sequence chain on a glass surface. FIG. 4C shows that photocrosslinked cross-junctions will only be created within the illuminated areas.

FIGS. 5-12 are reaction schemes for synthesis of template nucleic acids and synthesis of a nucleic acid sequence therefrom for a template with 8-junctions or blocking domains (FIG. 5), 7-junctions (FIG. 6), 6-junctions (FIG. 7), 5-junctions (FIG. 8), 4-junctions (FIG. 9), 3-junctions (FIG. 10), 2-junctions (FIGS. 11) and 1-junction (FIG. 12). First, template strands were annealed together. Then, a cross junction synthesis reaction was performed with a primer as depicted. Finally, PCR was performed to amplify the synthesis product into a double-stranded amplicon.

FIG. 13A shows Real-time qPCR amplification of the synthesis depicted in FIGS. 5-12. PCR reactions corresponding to reactions 1-8 presented above (FIGS. 5-12) were monitored on a real-time PCR machine using an intercalating dye. All 8 cross junction synthesis reactions were diluted 1000× (final) into PCR solutions and showed amplification around the same cycle. As a control, 8 reactions with all components except dNTPs were also mixed with the PCR master mix to check that amplicons did not result from the PCR step and template strands alone.

FIG. 13B is a PAGE denaturing gel showing the 8 synthesis products of expected length.

FIG. 13C shows Sanger sequencing results for the synthesis from the 8-junction template (FIG. 5) matched the programmed sequence in both directions.

FIGS. 14-16 are reaction schemes for synthesis of template nucleic acids by light-directed 2-junction (FIG. 14), 4-junction (FIGS. 15) and 6-junction (FIG. 16) concatemer formation followed by synthesis. A biotinylated probe sequence was attached to a glass slide through a biotin-streptavidin interaction. Template strands were introduced iteratively to form a template containing three strands, and then cross junction synthesis was performed with the rev a primer. After dilution and PCR, the double-stranded product was formed.

FIG. 17 is a PAGE denaturing gel showing the readout of PCR products of the synthesis depicted in FIGS. 5-12.

FIG. 18 is a schematic representation of an exemplary combinatorial cross-junction synthesis. The example above shows a combinatorial library formed by the inclusion of three possible options for the second-from-left template strand and two options for the second-from-right template strand. By including all possibilities in the annealing reaction, a library of concatemers is formed, from which a library of synthesized sequences can be formed.

FIG. 19A is a schematic representation of an embodiment of synthesis of a template nucleic acid and synthesis of a nucleic acid strand therefrom where the full length template is assembled and added to the synthesis reaction. FIG. 19B shows results of synthesis scheme shown in FIG. 19A under different conditions as specified under each lane. Each band represents the extension steps. It can be seen that lanes 1 and 7 have highest efficiency.

FIG. 20A-20B are bar graphs showing Cq values of qPCR reactions amplifying products of synthesis across 5-junctions performed under different salt and polymerase conditions with 10 minute (FIG. 20A) or 20 (FIG. 20B) minute incubations.

FIG. 21A shows a schematic representation of an exemplary embodiments nucleic acid synthesis method where guide strands are added consecutively to the reaction mixture during synthesis to assemble the full length template nucleic acid strand. FIG. 21B shows expected sequencing results (top) compared to Sanger sequencing results of PCR amplified product of synthesis across 5-junctions performed using an embodiment of the exemplary method depicted in FIG. 21.

### DETAILED DESCRIPTION

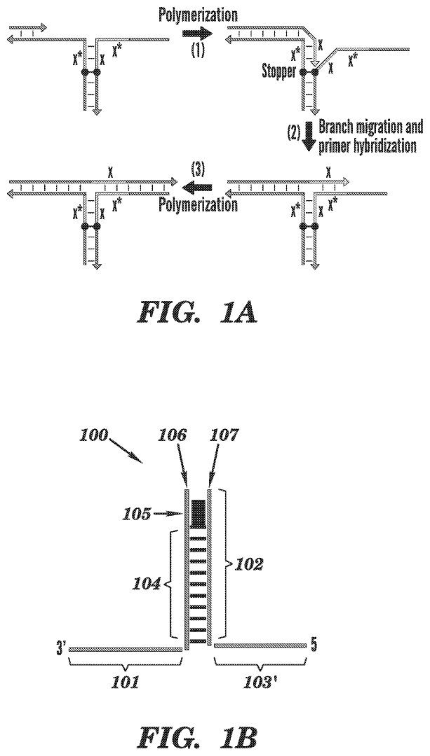

The fundamental strategy for cross-junction synthesis is depicted in FIG. 1. Shown are two guide strands (also referred to as template strands) that have been hybridized together to form a junction, and a primer that has bound in front of that junction on the first template strand (left). A strand displacing polymerase is used to copy the x domain (also referred to as a junction domain herein) until it reaches a stopper (shown in black and also referred to as a blocking domain herein). Afterwards, the new and old x domains compete in a random walk branch migration process (Lee et al., 1970). Ultimately, the new x domain can bind to the exposed x* domain on the second template strand (right, also referred to as a synthesis region herein), thus successfully crossing the junction. Polymerization can then continue on the second template strand, copying along a new backbone.

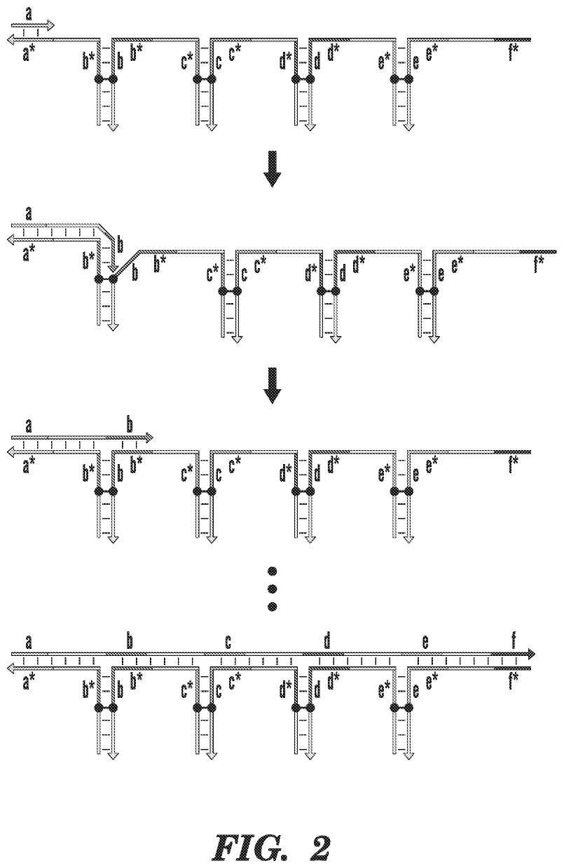

Accordingly, in one aspect provided herein is a nucleic acid template. As used herein, the “nucleic acid template” or a “template nucleic acid” refers to a nucleic acid comprising at least two guide strands (106 and 107) forming at least one cross-junction (100). Without limitations, the nucleic acid template can comprise any number of cross-junctions (100), i.e., the nucleic acid template can comprise any number for guide strands for forming the desired number of cross-junctions. Multiple guide strands can be hybridized to make up a full nucleic acid template that promotes synthesis of a desired nucleic acid sequence. FIGS. 1A-2 provide guidance for the structure of an exemplary nucleic acid template based on secondary structure and hybridization of the first and second guide strand. The nucleic acid template can be designed based on the desired nucleotide sequence to be synthesized.

As disclosed herein, the nucleic acid template comprises one or cross-junctions. Generally, each cross-junction comprises in a 3′ to 5′ direction a first guide strand and a second guide strand. The first guide strand comprises in a 3′ to 5′ direction optionally a first blocking region, optionally a first junction domain, a first synthesis region, a second synthesis region, a second junction domain and a second blocking region. The second guide strand comprises in a 3′ to 5′ direction a first blocking region, a first junction domain, a first synthesis region, a second synthesis region, optionally a second junction domain and optionally a second blocking region. The second blocking region of the first guide strand and the first blocking region of the second guide strand together form a blocking domain that blocks strand displacement activity of a polymerase. The second junction domain of the first guide strand comprises a nucleotide sequence that is substantially complementary to a nucleic acid sequence of the first junction domain of the second guide strand to form a double-stranded region. The second junction domain of the first guide strand comprises a nucleotide sequence substantially identical to a nucleotide sequence of the first synthesis region of the second guide strand.

When the nucleic acid template comprises two or more cross-junctions, the first guide strand of a first cross junction can be a second guide strand of a second cross-junction. Likewise, the second guide stand of a first cross junction can be a first guide strand of a second cross-junction.

It is noted that a guide strand described herein can comprise a barcode domain. For example, the second guide strand of a junction can comprise barcode domain on at the 5′-terminus. In some embodiments, one of the first or second synthesis regions of a guide strand comprises a barcode domain.

As used herein, a “barcode domain,” refers to the part of a strand, e.g., a guide strand that comprises a nucleic acid sequence that represents information or data. Non-limiting examples include spatial information, sequences, molecule sequences, experiment/batch number, binary codes, random barcodes to create unique molecular identifiers (UMI's), or any combination thereof. The barcode domain sequence can be predetermined by a barcode library. The barcode domain can be any suitable sequence. A barcode domain can be assigned a bit value. For example, each barcode domain can be independently assigned a bit value. It is noted that bit values are not limited to 0 and 1. In some embodiments, the barcode domain sequence can be 0 nucleotides in length. For example, lack of a barcode sequence can provide information (e.g, a 0 bit value is assigned to the absence of a barcode and a 1 bit value can be represented by a barcode domain of 1 or more nucleotides in length).

In some embodiments, the barcode domain is immobilized on a substrate surface. In some embodiments, the barcode is immobilized in a predetermined pattern. In some embodiments the barcode domain represents spatial information.

A guide strand described herein can also comprise a primer sequence. For example, the first guide strand of a junction can comprise a primer sequence at the 3′-terminus. In another non-limiting example, the second guide strand of a junction can comprise a primer sequence at the 5′-terminus.

In some embodiments, the nucleic acid template comprises a first guide strand and a second guide strand. The first guide strand comprises in 3′ to 5′ direction a first synthesis region, a second synthesis region, a first junction domain, and a first blocking region. The second guide strand comprises in 3′ to 5′ direction a second blocking region, a second junction domain and a third synthesis region. The first junction domain comprises a nucleotide sequence substantially identical to a nucleotide sequence of the third synthesis region, and the first junction domain and second junction domain are substantially complementary to each other and form a double-stranded region. The first blocking region and the second blocking region together form a first blocking domain that blocks strand displacement activity of a polymerase.

In some embodiments, the second guide strand further comprises a fourth synthesis region at the 5′ end of the third synthesis region.

As used herein, the terms “guide strand” or “template strand” are used interchangeably to refer to part of the nucleic acid template and serves as the basic building block of the nucleic acid template. Several guide strands can be combined to make up the nucleic acid template used for synthesizing a desired output nucleic acid sequence. The guide strand can for example, comprise in the 3′ to 5′ direction a first synthesis region, a second synthesis region, a first junction domain, and a first blocking region. The guide strand can also comprise in 3′ to 5′ direction a second blocking region, a second junction domain and a third synthesis region. For reference, several exemplary guide strands are depicted in FIG. 2. A set of guide strands (template strands) can be used to assemble the full length template nucleic acid used for nucleic acid synthesis.

As used herein, a “synthesis region” refers to part of a template strand (guide strand) that is to be copied to the nucleic acid sequence being synthesized. Without limitation, each synthesis region can independently comprise any desired nucleotide sequence. In other words, each synthesis region can be independently of any length. For example, each synthesis region can be one-nucleotide to few thousand nucleotides in length. In some embodiments, each synthesis region is 1,000-nucleotides or less, 750-nucleotides or less, 500-nucleotides or less, 400-nucleotides or less, 300-nucleotides or less, 250-nucleotides or less, 200-nucleotides or less, 150-nucleotides or less, 100-nucleotides or less in length or 50-nucleootide or less in length. Without limitations, a synthesis region can be just one nucleotide.

Without limitation, the double-stranded region of the cross junction can independently comprise any desired nucleotide sequence or number of base-pairs. In other words, the double-stranded region can be independently of any length. For example, each double-stranded region can be one base pair to tens of base pairs in length. In some embodiments, double-stranded region can be independently at least one, at least two, at least three, at least four, at least five, at least six, at least seven, at least eight, at least nine or at least ten nucleotides or base pairs in length.

As used herein, a “junction domain” refers to part of a guide strand that can hybridize with part of a second guide strand to form a duplex, and a nucleic acid sequence produced from a nucleic acid template comprising the guide strand comprises: (i) a sequence complimentary to the nucleotide sequence of the junction domain; or (ii) a sequence complimentary to the reverse complement of the nucleotide sequence of the junction domain. In other words, a nucleic acid sequence produced from a nucleic acid template comprising the guide strand comprises either a sequence that synthesized from the junction domain sequence or the reverse complement of the junction domain sequence.

Without limitation, each junction domain can independently comprise any desired nucleotide sequence or number of nucleotides. In other words, each junction domain can be independently of any length. For example, each junction domain can be one nucleotide to tens of nucleotide in length. In some embodiments, each junction domain can be independently one, two, three, four, five, six, seven, eight, nine or ten nucleotides in length.

As noted above, junction domain of a first guide strand can hybridize with a junction domain of a second domain to form a double-stranded structure. Without limitations, each duplex region can independently comprise any desired number of base-pairs. In other words, each duplex region can be independently of any length. For example, each duplex region can be one base pair to tens of base pairs in length. In some embodiments, each duplex region can be independently one, two, three, four, five, six, seven, eight, nine or ten nucleotides or base pairs in length.

As used herein, a “blocking domain” refers to part of the nucleic acid template that can prevent or stop a polymerase from continuing moving along the backbone of a template strand and/or copying the template strand. For example, the blocking domain can be a double-stranded region having high stability, e.g., a super-stable double-stranded region such that a polymerase is unable to separate the two strands. Methods for forming super-stable double-stranded nucleic acids are well known in the art and amenable to the invention. For example, the blocking domain can comprise one or more modified nucleotides known in the art for increasing Tₘ of double-stranded nucleic acids. Exemplary such nucleotides include, but are not limited locked nucleic acids (LNAs), 2′-O-methoxy-ethyl (2′-MOE) nucleotides, 2,6-diamopurine, G-clamp (an analog of C having 4 hydrogen bonds) and guanidinium G-clamp nucleotides, and the like.

In some embodiments, the blocking domain can comprise covalent cross-linking of two guide strands. It is noted that covalent cross-linking can comprise the situation where the nucleotide at the 5′-terminus of the first guide strand is cross-linked to the nucleotide at 3′-end of the second template strand, e.g., via an oligonucleotide or a single nucleic acid backbone linkage (e.g., phosphodiester bond). Generally, covalent cross-linking of two guide strands excludes which strand can lead to one continuous sequence from the two guide strands. Thus, in some embodiments, at least one (or both) of the nucleotides involved in the covalent cross-linkage is not a terminus nucleotide. In other words, at least one (or both) of the nucleotides involved in the cross-linking is not the first or last nucleotide of the template strand.

Without limitation, each blocking domain can independently comprise any desired nucleotide sequence or number of base-pairs. In other words, each blocking region can be independently of any length. For example, each blocking domain can be one base pair to tens of base pairs in length. In some embodiments, each blocking domain can be independently one, two, three, four, five, six, seven, eight, nine or ten nucleotides or base pairs in length.

In some embodiments, at least one of the blocking domain comprises a poly-monomer stretch. For example, the blocking domain comprises a stretch of polyA, polyT, polyC or polyG.

As used herein, a “blocking region” refers to a part of a template strand that along with a second template strand forms the blocking domain. Without limitations, the blocking region can independently comprise any desired nucleotide sequence or number of nucleotides. In other words, each blocking region can be independently of any length. For example, each blocking region can be one nucleotide to tens of nucleotides in length. In some embodiments, each blocking region can be independently one, two, three, four, five, six, seven, eight, nine or ten nucleotides in length. It is noted that a blocking region can be a single nucleotide.

In some embodiments, at least one blocking region within a blocking domain comprises a modified nucleotide that comprises a modification for cross-linking with another blocking region in the blocking domain.

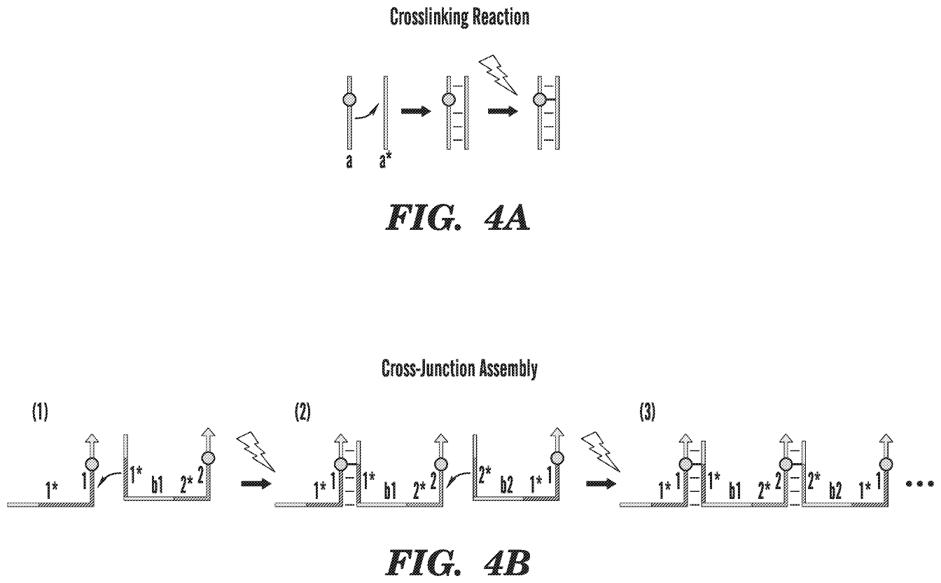

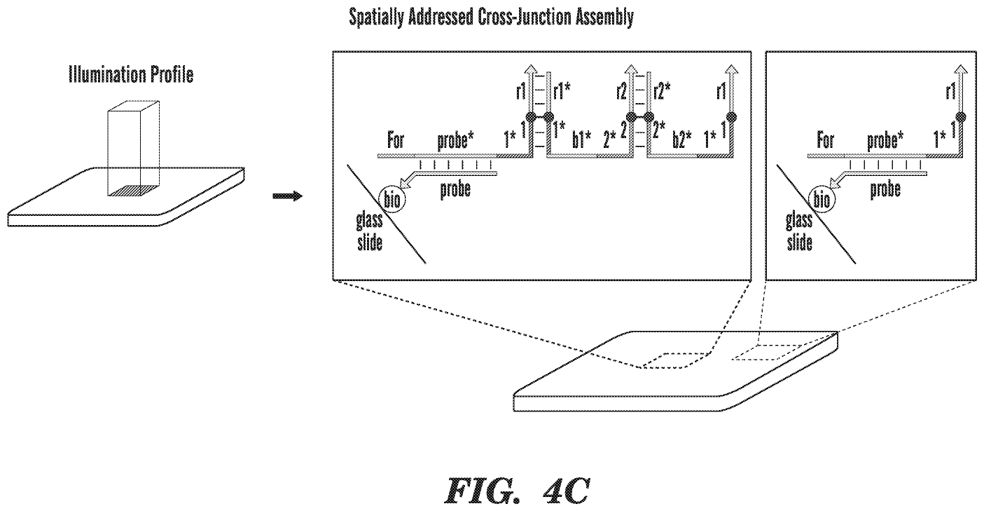

As used herein, the term “cross-linking segment,” refers to a part of the nucleic acid template that comprises a modified nucleotide that crosslinks to another oligonucleotide upon exposure to phase-changing stimulus (e.g. ultraviolet light, temperature change, pH change, etc.) and permits the assembly of the full nucleic acid template sequence. FIG. 4A provides an example of the crosslinking segments of the nucleic acid template.

Monomers and modified nucleotides for cross-linking of oligonucleotides are well known in the art. For example, photoreactive nucleotides are well known in the art for cross-linking oligonucleotides. Accordingly, in some embodiments, a blocking region can comprise a photoreactive nucleotide, i.e., a nucleotide with a photoreactive group. Exemplary photoreactive nucleotides include, but are not limited to, 3-Cyanovinylcarbazole (CNVK) nucleotide; 5-bromo deoxycytosine; 5-iodo deoxycytosine; 5-bromo deoxyurdine; 5-iodo deoxyuridine; and nucleotides comprising an aryl azide (AB-dUMP), benzophenone (BP-dUMP), perfluorinated aryl azide (FAB-dUMP) or diazirine (DB-dUMP). In some embodiments, a blocking region comprises CNVK as the photoreactive nucleotide.

In some embodiments of any of the aspects, the cross-linking segment comprises 3-cyanovinylcarbozole.

For example, the first and second blocking regions can be covalently linked to each other. Accordingly, in some embodiment, the first or the second blocking region comprises a cross-linking segment with the other blocking region to form the first blocking domain. For example, the cross-linking segment can comprise 3-cyanovinylcabozole for cross-linking.

Exemplary secondary structure of the nucleic acid template and guide strands that make up the nucleic acid template are shown in FIGS. 1A-2. The secondary structure allows for the DNA polymerase to displace from the first guide strand onto the second guide strand, third guide strand, fourth guide strand, fifth guide strand, etc. until the output nucleic acid sequence is achieved. Different structure prediction models can produce different predicted structures, and even the same model can produce different predicted structures if different baseline parameter are used, e.g., temperature, ionic strength, etc.

That said, when a change of one nucleotide in a base-paired structure of a guide strand within the nucleic acid template is accompanied by a compensatory change in the complementary nucleotide that maintains the ability to base pair, the structure, and thereby the function of the guide strand can be maintained. That is, some guide strands can tolerate some degree of sequence change and still retain DNA polymerase displacement activity. Furthermore, a truncated or partial sequence of a nucleic acid template or guide strand as described herein can also retain displacement activity provided that the truncation does not alter intramolecular base-pairing necessary for the secondary structure of the guide strand or nucleic acid template. The Examples herein provide working demonstration of the nucleic acid templates that specifically displace the DNA polymerase.

It is contemplated that the reverse, complement, reverse complement, or truncated sequences of the nucleic acid templates described herein can also maintain secondary structure of the template. Secondary structure models can be used to predict the stability of the guide strands in the nucleic acid template. The maintenance or improvement in modified guide strands to produce a nucleic acid output sequence, e.g. a compensatory or non-compensatory change can be made on the basis of predicted structure should be tested experimentally.

In some embodiments of any of the aspects, one or more regions of the nucleic acid template utilizes a 3-letter code. As used herein, a “3-letter code” means the region comprises only three of the four nucleobases, i.e., only three of adenine, thymine/uracil, guanine, and cytosine, or modified versions thereof. For example, a 3-letter code can comprise or consists of nucleobases selected from one of the following:

(i) adenine, thymine/uracil, and guanine;

(ii) adenine, thymine/uracil, and cytosine;

(iii) adenine, guanine, and cytosine; or

(iv) thymine/uracil, guanine, and cytosine.

In some embodiments of any of the aspects described herein, the nucleic acid template and/or the synthesized nucleic acid sequence comprises a nucleic acid modification. For example, at least one of a synthesis region, a junction domain, and/or a blocking domain can comprise a nucleic acid modification. Exemplary nucleic acid modifications include, but are not limited to, nucleobase modifications, sugar modifications, inter-sugar linkage modifications, conjugates (e.g., ligands), and any combinations thereof.

Exemplary modified nucleobases include, but are not limited to, inosine, xanthine, hypoxanthine, nubularine, isoguanisine, tubercidine, and substituted or modified analogs of adenine, guanine, cytosine and uracil, such as 2-aminoadenine, 6-methyl and other alkyl derivatives of adenine and guanine, 2-propyl and other alkyl derivatives of adenine and guanine, 5-halouracil and cytosine, 5-propynyl uracil and cytosine, 6-azo uracil, cytosine and thymine, 5-uracil (pseudouracil), 4-thiouracil, 5-halouracil, 5-(2-aminopropyl)uracil, 5-amino allyl uracil, 8-halo, amino, thiol, thioalkyl, hydroxyl and other 8-substituted adenines and guanines, 5-trifluoromethyl and other 5-substituted uracils and cytosines, 7-methylguanine, 5-substituted pyrimidines, 6-azapyrimidines and N-2, N-6 and O-6 substituted purines, including 2-aminopropyladenine, 5-propynyluracil and 5-propynylcytosine, dihydrouracil, 3-deaza-5-azacytosine, 2-aminopurine, 5-alkyluracil, 7-alkylguanine, 5-alkyl cytosine,7-deazaadenine, N6, N6-dimethyladenine, 2,6-diaminopurine, 5-amino-allyl-uracil, N3-methyluracil, substituted 1,2,4-triazoles, 2-pyridinone, 5-nitroindole, 3-nitropyrrole, 5-methoxyuracil, uracil-5-oxyacetic acid, 5-methoxycarbonylmethyluracil, 5-methyl-2-thiouracil, 5-methoxycarbonylmethyl-2-thiouracil, 5-methylaminomethyl-2-thiouracil, 3-(3-amino-3 carboxypropyl)uracil, 3-methylcytosine, 5-methylcytosine, N⁴-acetyl cytosine, 2-thiocytosine, N6-methyladenine, N6-isopentyladenine, 2-methylthio-N6-isopentenyladenine, N-methylguanines, or O-alkylated bases. Further purines and pyrimidines include those disclosed in U.S. Pat. No. 3,687,808, those disclosed in the Concise Encyclopedia of Polymer Science and Engineering, pages 858-859, Kroschwitz, J. I., ed. John Wiley & Sons, 1990, and those disclosed by Englisch et al., Angewandte Chemie, International Edition, 1991, 30, 613.

In some embodiments, modified nucleobase can be selected from the group consisting of inosine, xanthine, hypoxanthine, nubularine, isoguanisine, tubercidine, 2-(halo)adenine, 2-(alkyl)adenine, 2-(propyl)adenine, 2-(amino)adenine, 2-(aminoalkyll)adenine, 2-(aminopropyl)adenine, 2-(methylthio)-N⁶-(isopentenyl)adenine, 6-(alkyl)adenine, 6-(methyl)adenine, 7-(deaza)adenine, 8-(alkenyl)adenine, 8-(alkyl)adenine, 8-(alkynyl)adenine, 8-(amino)adenine, 8-(halo)adenine, 8-(hydroxyl)adenine, 8-(thioalkyl)adenine, 8-(thiol)adenine, N⁶-(isopentyl)adenine, N⁶-(methyl)adenine, N⁶, N⁶-(dimethyl)adenine, 2-(alkyl)guanine,2-(propyl)guanine, 6-(alkyl)guanine, 6-(methyl)guanine, 7-(alkyl)guanine, 7-(methyl)guanine, 7-(deaza)guanine, 8-(alkyl)guanine, 8-(alkenyl)guanine, 8-(alkynyl)guanine, 8-(amino)guanine, 8-(halo)guanine, 8-(hydroxyl)guanine, 8-(thioalkyl)guanine, 8-(thiol)guanine, N-(methyl)guanine, 2-(thio)cytosine, 3-(deaza)-5-(aza)cytosine, 3-(alkyl)cytosine, 3-(methyl)cytosine, 5-(alkyl)cytosine, 5-(alkynyl)cytosine, 5-(halo)cytosine, 5-(methyl)cytosine, 5-(propynyl)cytosine, 5-(propynyl)cytosine, -(trifluoromethyl)cytosine, 6-(azo)cytosine, N⁴-(acetyl)cytosine, 3-(3-amino-3-carboxypropyl)uracil, 5-ethynyl-2′-deoxyuridine, 2-(thio)uracil, 5-(methyl)-2-(thio)uracil, 5-(methylaminomethyl)-2-(thio)uracil, 4-(thio)uracil, 5-(methyl)-4-(thio)uracil, 5-(methylaminomethyl)-4-(thio)uracil, 5-(methyl)-2,4-(dithio)uracil, 5-(methylaminomethyl)-2,4-(dithio)uracil, 5-(2-aminopropyl)uracil, 5-(alkyl)uracil, 5-(alkynyl)uracil, 5-(allylamino)uracil, 5-(aminoallyl)uracil, 5-(aminoalkyl)uracil, 5-(guanidiniumalkyl)uracil, 5-(1,3-diazole- 1-alkyl)uracil, 5-(cyanoalkyl)uracil, 5-(dialkylaminoalkyl)uracil, 5-(dimethylaminoalkyl)uracil, 5-(halo)uracil, 5-(methoxy)uracil, uracil-5-oxyacetic acid, 5-(methoxycarbonylmethyl)-2-(thio)uracil, 5-(methoxycarbonyl-methyl)uracil, 5-(propynyl)uracil, 5-(propynyl)uracil, 5-(trifluoromethyl)uracil, 6-(azo)uracil, dihydrouracil, N³-(methyl)uracil, 5-uracil (i.e., pseudouracil), 2-(thio)pseudouraci1,4-(thio)pseudouraci1,2,4-(dithio)psuedouracil, 5-(alkyl)pseudouracil, 5-(methyl)pseudouracil, 5-(alkyl)-2-(thio)pseudouracil, 5-(methyl)-2-(thio)pseudouracil, 5-(alkyl)-4-(thio)pseudouracil, 5-(methyl)-4-(thio)pseudouracil, 5-(alkyl)-2,4-(dithio)pseudouracil, 5-(methyl)-2,4-(dithio)pseudouracil, 1-substituted pseudouracil, 1-substituted 2(thio)-pseudouracil, 1-substituted 4-(thio)pseudouracil, 1-substituted 2,4-(dithio)pseudouracil, 1-(aminocarbonylethylenyl)-pseudouracil, 1-(aminocarbonylethylenyl)-2(thio)-pseudouracil, 1-(aminocarbonylethylenyl)-4-(thio)pseudouracil, 1-(aminocarbonylethylenyl)-2,4-(dithio)pseudouracil, 1-(aminoalkylaminocarbonylethylenyl)-pseudouracil, 1-(aminoalkylamino-carbonylethylenyl)-2(thio)-pseudouracil, 1-(aminoalkylaminocarbonylethylenyl)-4-(thio)pseudouracil, 1-(aminoalkylaminocarbonylethylenyl)-2,4-(dithio)pseudouracil, 1,3-(diaza)-2-(oxo)-phenoxazin- 1-yl, 1-(aza)-2-(thio)-3-(aza)-phenoxazin- 1-yl, 1,3-(diaza)-2-(oxo)-phenthiazin-1-yl, 1-(aza)-2-(thio)-3-(aza)-phenthiazin- 1-yl, 7-substituted 1,3-(diaza)-2-(oxo)-phenoxazin- 1-yl, 7-substituted 1-(aza)-2-(thio)-3-(aza)-phenoxazin- 1-yl, 7-substituted 1,3-(diaza)-2-(oxo)-phenthiazin- 1-yl, 7-substituted 1-(aza)-2-(thio)-3-(aza)-phenthiazin-1-yl, 7-(aminoalkylhydroxy)- 1,3-(diaza)-2-(oxo)-phenoxazin- 1-yl, 7-(aminoalkylhydroxy)-1-(aza)-2-(thio)-3-(aza)-phenoxazin- 1-yl, 7-(aminoalkylhydroxy)- 1,3-(diaza)-2-(oxo)-phenthiazin- 1-yl, 7-(aminoalkylhydroxy)- 1-(aza)-2-(thio)-3-(aza)-phenthiazin-1-yl, 7-(guanidiniumalkylhydroxy)- 1,3-(diaza)-2-(oxo)-phenoxazin- 1-yl, 7-(guanidiniumalkylhydroxy)-1-(aza)-2-(thio)-3-(aza)-phenoxazin- 1-yl, 7-(guanidiniumalkyl-hydroxy)-1,3-(diaza)-2-(oxo)-phenthiazin- 1-yl, 7-(guanidiniumalkylhydroxy)- 1-(aza)-2-(thio)-3-(aza)-phenthiazin- 1-yl, 1,3,5-(triaza)-2,6-(dioxa)-naphthalene, inosine, xanthine, hypoxanthine, nubularine, tubercidine, isoguanisine, inosinyl, 2-aza-inosinyl, 7-deaza-inosinyl, nitroimidazolyl, nitropyrazolyl, nitrobenzimidazolyl, nitroindazolyl, aminoindolyl, pyrrolopyrimidinyl, 3-(methyl)isocarbostyrilyl, 5-(methyl)isocarbostyrilyl, 3-(methyl)-7-(propynyl)isocarbostyrilyl, 7-(aza)indolyl, 6-(methyl)-7-(aza)indolyl, imidizopyridinyl, 9-(methyl)-imidizopyridinyl, pyrrolopyrizinyl, isocarbostyrilyl, 7-(propynyl)isocarbostyrilyl, propynyl-7-(aza)indolyl, 2,4,5-(trimethyl)phenyl, 4-(methyl)indolyl, 4,6-(dimethyl)indolyl, phenyl, napthalenyl, anthracenyl, phenanthracenyl, pyrenyl, stilbenyl, tetracenyl, pentacenyl, difluorotolyl, 4-(fluoro)-6-(methyl)benzimidazole, 4-(methyl)benzimidazole, 6-(azo)thymine, 2-pyridinone, 5-nitroindole, 3-nitropyrrole, 6-(aza)pyrimidine, 2-(amino)purine, 2,6-(diamino)purine, 5-substituted pyrimidines, N²-substituted purines, N⁶-substituted purines, 0⁶-substituted purines, substituted 1,2,4-triazoles, and any 0-alkylated or N-alkylated derivatives thereof.

Exemplary sugar modifications include, but are not limited to, 2′-Fluoro, 3′-Fluoro, 2′-OMe, 3′-OMe, 2′-deoxy modifications, and acyclic nucleotides, e.g., peptide nucleic acids (PNA), unlocked nucleic acids (UNA) or glycol nucleic acid (GNA).

In some embodiments, a nucleic acid modification can include replacement or modification of an inter-sugar linkage. Exemplary inter-sugar linkage modifications include, but are not limited to, phosphotriesters, methylphosphonates, phosphoramidate, phosphorothioates, methylenemethylimino, thiodiester, thionocarbamate, siloxane, N,N′-dimethylhydrazine (—CH2-N(CH3)-N(CH3)-), amide-3 (3′-CH₂—C(═O)—N(H)-5′) and amide-4 (3′-CH₂—N(H)—C(═O)-5′), hydroxylamino, siloxane (dialkylsiloxxane), carboxamide, carbonate, carboxymethyl, carbamate, carboxylate ester, thioether, ethylene oxide linker, sulfide, sulfonate, sulfonamide, sulfonate ester, thioformacetal (3′-S—CH₂—O-5′), formacetal (3′—O—CH₂—O-5′), oxime, methyleneimino, methykenecarbonylamino, methylenemethylimino (MMI, 3′-CH₂—N(CH₃)—O-5′), methylenehydrazo, methylenedimethylhydrazo, methyleneoxymethylimino, ethers (C3′-O—C5′), thioethers (C3′-S—C5′), thioacetamido (C3′-N(H)—C(═O)—CH₂—S—C5′, C3′-O—P(O)—O—SS—C5′, C3′-CH₂—NH—NH—C5′, 3′-NHP(O)(OCH₃)—O-5′ and 3′-NHP(O)(OCH₃)—O-5′

In some embodiments, nucleic acid modifications can include peptide nucleic acids (PNA), bridged nucleic acids (BNA), morpholinos, locked nucleic acids (LNA), glycol nucleic acids (GNA), threose nucleic acids (TNA), or any other xeno nucleic acids (XNA) described in the art.

In some embodiments of the various aspects, the nucleic acid template, a guide strand, and/or the synthesized nucleic acid sequence can be modified on the 3′- and/or 5′-end. For example, a label, fluorophore, tag, or a cap can be added to the 3′ and/or 5′-end of the nucleic acid template, the guide strand, and/or the synthesized nucleic acid sequence.

In some embodiments of the various aspects, the nucleic acid template, a guide strand, and/or the synthesized nucleic acid sequence can be modified with a linker or spacer, e.g., at an internal position, on the 3′- and/or 5′-end. Without wishing to be bound by a theory, the linker or spacer can be used for linking the nucleic acid template, the guide strand, and/or the synthesized nucleic acid sequence with a moiety, such as a solid support or label. In some embodiments, the linker or spacer can be selected from the group consisting of photocleavable linkers, hydrolyzable linkers, redox cleavable linkers, phosphate -based cleavable linkers, acid cleavable linkers, ester-based cleavable linkers, peptide-based cleavable linkers, and any combinations thereof. In some embodiments, the cleavable linker can comprise a disulfide bond, a tetrazine-trans-cyclooctene group, a sulfhydryl group, a nitrobenzyl group, a nitoindoline group, a bromo hydroxycoumarin group, a bromo hydroxyquinoline group, a hydroxyphenacyl group, a dimethozybenzoin group, or any combinations thereof.

Any art-recognized photocleavable linker can be used. In some embodiments, the cleavable linker can comprise a photocleavable linker. Generally, photocleavable linkers contain a photolabile functional group that is cleavable upon exposure to a light source (e.g., UV light) or specific wavelength. Non-limiting examples of photocleavable spacers can be found, for example, in U.S. Pat. Nos. 6,589,736 B1; 7,622,279 B2; 9,371,348 B2; 7,547,530 B2; and 7,057,031 B2; and PCT Publication No. WO2014200767, contents of all of which are incorporated herein by reference in their entirety.

In some embodiments of the various aspects, the nucleic acid template, a guide strand, and/or the synthesized nucleic acid sequence can be modified with a detectable label, e.g., at an internal position, on the 3′- and/or 5′-end. Without wishing to be bound by a theory, such a detectable label can facilitate detection. As used herein, the term “detectable label” refers to a composition capable of producing a detectable signal indicative of the presence of a target. Detectable labels include any composition detectable by spectroscopic, photochemical, biochemical, immunochemical, electrical, optical or chemical means. Suitable labels include fluorescent molecules, radioisotopes, nucleotide chromophores, enzymes, substrates, chemiluminescent moieties, bioluminescent moieties, and the like. As such, a label is any composition detectable by spectroscopic, photochemical, biochemical, immunochemical, electrical, optical or chemical means.

A wide variety of fluorescent reporter dyes are known in the art. Typically, the fluorophore is an aromatic or heteroaromatic compound and can be a pyrene, anthracene, naphthalene, acridine, stilbene, indole, benzindole, oxazole, thiazole, benzothiazole, cyanine, carbocyanine, salicylate, anthranilate, coumarin, fluorescein, rhodamine or other like compound.

Exemplary fluorophores include, but are not limited to, 1,5 IAEDANS; 1,8-ANS ; 4-Methylumbelliferone; 5-carboxy-2,7-dichlorofluorescein; 5-Carboxyfluorescein (5-FAM); 5-Carboxynapthofluorescein (pH 10); 5-Carboxytetramethylrhodamine (5-TAMRA); 5-FAM (5-Carboxyfluorescein); 5-Hydroxy Tryptamine (HAT); 5-ROX (carboxy-X-rhodamine); 5-TAMRA (5-Carboxytetramethylrhodamine); 6-Carboxyrhodamine 6G; 6-CR 6G; 6-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Aequorin (Photoprotein); Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC, AMCA-S; AMCA (Aminomethylcoumarin); AMCA-X; Aminoactinomycin D; Aminocoumarin; Anilin Blue; Anthrocyl stearate; APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); BCECF (high pH); BCECF (low pH); Berberine Sulphate; Beta Lactamase; BFP blue shifted GFP (Y66H); BG-647; Bimane; Bisbenzamide; Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy F1; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Green-1 Ca²⁺ Dye; Calcium Green-2 Ca²⁺; Calcium Green-5N Ca²⁺; Calcium Green-C18 Ca²⁺; Calcium Orange; Calcofluor White; Carboxy-X-rhodamine (5-ROX); Cascade Blue™; Cascade Yellow; Catecholamine; CFDA; CFP-Cyan Fluorescent Protein; Chlorophyll; Chromomycin A; Chromomycin A; CMFDA; Coelenterazine ; Coelenterazine cp; Coelenterazine f; Coelenterazine fcp; Coelenterazine h; Coelenterazine hcp; Coelenterazine ip; Coelenterazine O; Coumarin Phalloidin; CPM Methylcoumarin; CTC; Cy2™; Cy3.1 8; Cy3.5™; Cy3™; Cy5.1 8; Cy5.5™; Cy5™; Cy7™; Cyan GFP; cyclic AMP Fluorosensor (FiCRhR); d2; Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydorhodamine 123); Di-4-ANEPPS; Di-8-ANEPPS (non-ratio); DiA (4-Di-16-ASP); DIDS; Dihydorhodamine 123 (DHR); DiO (DiOC18(3)); DiR; DiR (DiIC18(7)); Dopamine; DsRed; DTAF; DY-630-NHS; DY-635-NHS; EBFP; ECFP; EGFP; ELF 97; Eosin; Erythrosin; Erythrosin ITC; Ethidium homodimer-1 (EthD-1); Euchrysin; Europium (III) chloride; Europium; EYFP; Fast Blue; FDA; Feulgen (Pararosaniline); FITC; FL-645; Flazo Orange; Fluo-3; Fluo-4; Fluorescein Diacetate; Fluoro-Emerald; Fluoro-Gold (Hydroxystilbamidine); Fluor-Ruby; FluorX; FM 1-43™; FM 4-46; Fura Red™ (high pH); Fura-2, high calcium; Fura-2, low calcium; Genacryl Brilliant Red B; Genacryl Brilliant Yellow 10GF; Genacryl Pink 3G; Genacryl Yellow 5GF; GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indodicarbocyanine (DiD); Indotricarbocyanine (DiR); Intrawhite Cf; JC-1; JO-JO-1; JO-PRO-1; LaserPro; Laurodan; LDS 751; Leucophor PAF; Leucophor SF; Leucophor WS; Lissamine Rhodamine; Lissamine Rhodamine B; LOLO-1; LO-PRO-1; Lucifer Yellow; Mag Green; Magdala Red (Phloxin B); Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; Nitrobenzoxadidole; Noradrenaline; Nuclear Fast Red; Nuclear Yellow; Nylosan Brilliant Iavin E8G; Oregon Green™; Oregon Green 488-X; Oregon Green™488; Oregon Green™ 500; Oregon Green™ 514; Pacific Blue; Pararosaniline (Feulgen); PE-Cy5; PE-Cy7; PerCP; PerCP-Cy5.5; PE-TexasRed (Red 613); Phloxin B (Magdala Red); Phorwite AR; Phorwite BKL; Phorwite Rev; Phorwite RPA; Phosphine 3R; PhotoResist; Phycoerythrin B [PE]; Phycoerythrin R [PE]; PKH26 ; PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Resorufin; RH 414; Rhod-2; Rhodamine; Rhodamine 110; Rhodamine 123; Rhodamine 5 GLD; Rhodamine 6G; Rhodamine B 540; Rhodamine B 200 ; Rhodamine B extra; Rhodamine BB; Rhodamine BG; Rhodamine Green; Rhodamine Phallicidine; Rhodamine Phalloidine; Rhodamine Red; Rhodamine WT; Rose Bengal; R-phycoerythrin (PE); red shifted GFP (rsGFP, S65T); S65A; S65C; S65L; S65T; Sapphire GFP; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SPQ (6-methoxy-N-(3-sulfopropyl)-quinolinium); Stilbene; Sulphorhodamine B can C; Sulphorhodamine G Extra; Tetracycline; Tetramethylrhodamine ; Texas Red™; Texas Red-X™ conjugate; Thiadicarbocyanine (DiSC3); Thiazine Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC (TetramethylRodamineIsoThioCyanate); True Blue; TruRed; Ultralite; Uranine B; Uvitex SFC; wt GFP; WW 781; XL665; X-Rhodamine; XRITC; Xylene Orange; Y66F; Y66H; Y66W; Yellow GFP; YFP; YO-PRO-1; YO-PRO-3; YOYO-1; and YOYO-3. Many suitable forms of these fluorescent compounds are available and can be used.

Other exemplary detectable labels include luminescent and bioluminescent markers (e.g., biotin, luciferase (e.g., bacterial, firefly, click beetle and the like), luciferin, and aequorin), radiolabels (e.g., 3H, 1251, 35S, 14C, or 32P), enzymes (e.g., galactosidases, glucorinidases, phosphatases (e.g., alkaline phosphatase), peroxidases (e.g., horseradish peroxidase), and cholinesterases), and calorimetric labels such as colloidal gold or colored glass or plastic (e.g., polystyrene, polypropylene, and latex) beads. Patents teaching the use of such labels include U.S. Pat. Nos. 3,817,837, 3,850,752, 3,939,350, 3,996,345, 4,277,437, 4,275,149, and 4,366,241, each of which is incorporated herein by reference.

Means of detecting such labels are well known to those of skill in the art. Thus, for example, radiolabels can be detected using photographic film or scintillation counters, fluorescent markers can be detected using a photo-detector to detect emitted light. Enzymatic labels are typically detected by providing the enzyme with an enzyme substrate and detecting the reaction product produced by the action of the enzyme on the enzyme substrate, and calorimetric labels can be detected by visualizing the colored label.

In some embodiments, the detectable label is a fluorophore or a quantum dot. Without wishing to be bound by a theory, using a fluorescent reagent can reduce signal-to-noise in the imaging/readout, thus maintaining sensitivity.

In some embodiments, a label can be configured to include a “smart label”, which is undetectable when conjugated with the nucleic acid template, the guide strand, and/or the synthesized nucleic acid sequence, but produces a color change when released.

Acrydite modifications can also be made to the nucleic acid template, a guide strand, and/or the synthesized nucleic acid sequence. Acrydite modifications can permit the oligonucleotides to be used in reactions with nucleophiles such as thiols (e.g, microarrays) or incorporated into gels (e.g, polyacrylamide). Accordingly, in some embodiments, the nucleic acid template, a guide strand, and/or the synthesized nucleic acid sequence comprises one or more acrydite nucleosides. The acrydite nucleoside can be at the 3′- end, 5-end, and/or at an internal position of the nucleic acid template, the guide strand, and/or the synthesized nucleic acid sequence.

Any modifications to the nucleic acid template, a guide strand, and/or the synthesized nucleic acid sequences provided herein that permit purification, extraction, quantification of expression, binding, electrophoresis, and the like, can be made.

As disclosed herein, the nucleic acid template can comprise any desired number of cross-junctions, i.e., the nucleic acid template can comprise more than two guide strands. For example, the nucleic acid template comprises two cross-junctions, e.g., the nucleic acid template comprises three guide strands. Thus, in some embodiments, the second guide strand further comprises at its 5′end in 3′ to 5′ direction a third junction domain and a third blocking region, and the nucleic acid template further comprises a third guide strand. The third guide strand comprises in 3′ to 5′ direction a fourth blocking region, a fourth junction domain and a fifth synthesis region. The third junction domain comprises a nucleotide sequence substantially identical to a nucleotide sequence of the fifth synthesis region, wherein the third junction domain and fourth junction domain are substantially complementary to each other and form a double-stranded region, and wherein the third blocking region and the fourth blocking region together form a second blocking domain that blocks strand displacement activity of a polymerase.

In some embodiments of any of the aspects, the third guide strand further comprises a sixth synthesis region at the 5′ end of the fifth synthesis region.

As disclosed herein, the third or the fourth blocking regions can be covalently linked to each other. Thus, one of the third or the fourth blocking region can comprise a cross-linking segment with the other blocking region to form the second blocking domain. In some embodiments, the cross-linking segment comprises 3-cyanovinylcabozole for cross-linking.

In some embodiments of any of the aspects, the nucleic acid template comprises three cross-junctions, e.g., the nucleic acid template comprises four guide strands. Accordingly, in some embodiments, the third guide strand further comprises at its 5′end in 3′ to 5′ direction a fifth junction domain and a fifth blocking region, and the nucleic acid template further comprises a fourth guide strand. The fourth strand comprises in 3′ to 5′ direction a sixth blocking region, a sixth junction domain and a seventh synthesis region. The fifth junction domain comprises a nucleotide sequence substantially identical to a nucleotide sequence of the seventh synthesis region, and the fourth junction domain and fifth junction domain are substantially complementary to each other and form a double-stranded region, and wherein the fifth blocking region and the sixth blocking region together form a third blocking domain that blocks strand displacement activity of a polymerase.

In some embodiments, the fourth guide strand further comprises an eighth synthesis region at the 5′ end of the sixth synthesis region.

As described herein, the fifth and sixth blocking regions can be covalently linked to each other. Accordingly, one of the fifth or the sixth blocking region can comprise a cross-linking segment with the other blocking region to form the third blocking domain. In some embodiments, the cross-linking segment comprises 3-cyanovinylcabozole for cross-linking.

In some embodiments of any of the aspects, the nucleic acid template comprises four cross-junctions, e.g., the nucleic acid template comprises five guide strands. Accordingly, in some embodiments, the fourth guide strand further comprises at its 5′end in 3′ to 5′ direction a seventh junction domain and a seventh blocking region, and the nucleic acid template further comprises a fifth guide strand. The fifth guide strand comprises in 3′ to 5′ direction an eighth blocking region, an eighth junction domain and a ninth synthesis region. The seventh junction domain comprises a nucleotide sequence substantially identical to a nucleotide sequence of the ninth synthesis region, and the seventh junction domain and eighth junction domain are substantially complementary to each other and form a double-stranded region, and wherein the seventh blocking region and the eighth blocking region together form a fourth blocking domain that blocks strand displacement activity of a polymerase.

As described herein, the seventh and the eighth blocking regions can be covalently linked to each other. Accordingly, one of the seventh or the eights blocking region can comprise a cross-linking segment with the other blocking region to form the fourth blocking domain. In some embodiments, the cross-linking segment comprises 3-cyanovinylcabozole.

In some embodiments, all guide strands in the nucleic acid template can comprise a substantially identical nucleotide sequence in the synthesis region in the 5′-side of the guide strands. In some embodiments, all guide strands in the nucleic acid template can comprise a substantially identical nucleotide sequence in the synthesis region in the 3′-side of the guide strands.

In some embodiments of any of the aspects, the nucleic acid template, guide strand, or portion thereof is immobilized, conjugated to, or linked to a solid support or a substrate. In some embodiments, the nucleic acid template, guide strand, or portion thereof is immobilized on a substrate surface. In some embodiments, the nucleic acid template, guide strand, or portion thereof represents information, date, or spatial information. It is noted that the nucleic acid template, guide strand, or portion thereof can be immobilized, conjugate to, or linked to the solid support or substrate covalently or non-covalently.

In some embodiments of any of the aspects, the nucleic acid template, guide strand, or portion thereof is immobilized in a predetermined pattern. In some embodiments of any of the aspects, the predetermined pattern is a geometric shape, a square, a circle, or triangle. In some embodiments of any of the aspects, the predetermined pattern comprises repeating elements. In some embodiments of any of the aspects, the predetermined pattern is asymmetrical or symmetrical. In some embodiments of any of the aspects, the predetermined pattern comprises spatial information and/or special information.

Without limitations, the solid support or substrate can exist in the form of a platform, column, filter or sheet, dish, a microfluidic capture device, capillary tube, electrochemical responsive platform, scaffold, cartridge, resin, matrix, bead, or another solid support known in the art. The substrate can also comprise a biological material. Biological materials are known in the art. Non-limiting examples of biological materials include tissues, tissue sections, engineered tissues, cells, patient derived cells, primary cells, organoids, extracellular matrix, 3D biological organs, dissociated cells, live cells, fixed cells, vesicles, droplets, liposomes, etc.

In some embodiments, the solid support or substrate comprises materials that include, but are not limited to, a polymer, metal, ceramic, gels, paper, or glass. The materials of the solid support can further comprise, as non-limiting examples, polystyrene, agarose, gelatin, alginate, iron oxide, stainless steel, gold nanobeads or particles, copper, silver chloride, polycarbonate, polydimethylsiloxane, polyethylene, acrylonitrile butadiene styrene, cyclo-olefin polymers or cyclo-olefin copolymers, or Sepharos™ resin.

In some embodiments, the substrate is a hydrogel. In some embodiments, the hydrogel is a compressed hydrogel. A hydrogel can be naturally occurring, derived from a natural source, or derived from a synthetic source. A hydrogel can be any water-swollen and cross-linked polymeric material produced by a reaction of one or more monomers. A hydrogel can be a polymeric material that is capable of expanding to retain a significant fraction of water within its structure without dissolving into the aqueous solution. A hydrogel can also be any shrinkable material, e.g., heat-shrinkable plastics, viscoelastic foam, memory foam.

In some embodiments, the solid support or substrate can further comprise a magnetoresponsive element such as a magnetoresponsive bead. In some embodiments, the magnetoresponsive element or bead is in the form of a sphere, cube, rectangle, cylinder, cone, or any other shape described in the art.

In some embodiments, the magnetoresponsive element comprises magnetite, iron (III) oxide, samarium-cobalt, terfenol-D, or any other magnetic element described in the art.

### Synthesis of Nucleic Acid Template

In another aspect, provided herein is a method for forming a nucleic acid template described herein. Generally, the method comprising annealing or hybridizing a first guide strand and a second guide strand. The first guide strand comprises in 3′ to 5′ direction a first synthesis region, a second synthesis region, a first junction domain, and a first blocking region. The second guide strand comprises in 3′ to 5′ direction a second blocking region, a second junction domain and a third synthesis region. The first junction domain comprises a nucleotide sequence substantially identical to a nucleotide sequence of the third synthesis region, and the first junction domain and second junction domain are substantially complementary to each other and form a double-stranded region, and wherein the first blocking region and the second blocking region together form a blocking domain that blocks strand displacement activity of a polymerase.

In some embodiments, the first or the second blocking region comprises a cross-linking segment with the other region to form the blocking domain and the method further comprises forming the cross-link.

The compositions and methods described herein can be used for synthesis of arbitrary length prescribed sequences. An exemplary embodiment is shown in FIG. 2. As shown in FIG. 2, by hybridizing multiple guide strands together, cross-junction synthesis reactions can be cascaded to form longer sequences. Each junction between guide strands shows the same domain motifs, whereby the strand domains copied on the 3′ end of the growing strand before reaching a stopper (b, c, d, e) can reach across the junction and bind to the exposed complementary sequence (b*, c*, d*, e*) on the next guide strand. Arbitrary sequences can be added in the template regions between the motif domains (shown in gray) to enable longer sequences to be assembled.

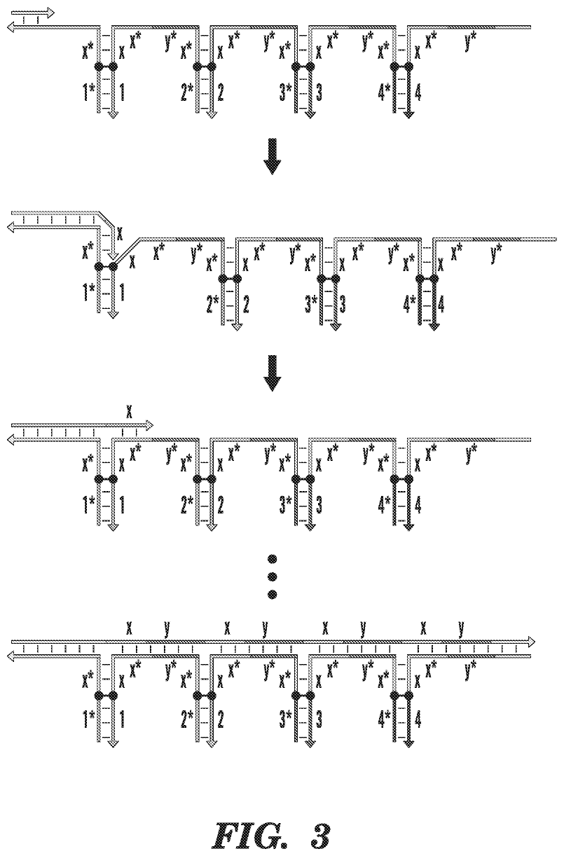

The compositions and methods described herein can be used for synthesis of polymeric sequences of prescribed length. For example, the compositions and methods described herein can be used for synthesizing specific numbers of repeated sequences through the use of programmable unique hybridization domains that fix the length of the template concatemer, which has been a long-standing challenge. An exemplary embodiment is shown in FIG. 3. As shown in FIG. 3, by harnessing the specificity of DNA self-assembly, unique binding sequences promote assemblies of prescribed length, even if the domains being synthesized are identical.

In some embodiments, cross-junctions can be assembled by utilizing a photoreactive inter-strand crosslinking base, which can serve the dual purpose of linking two cross-junction strands together and acting as a stopper for polymerase synthesis (FIG. 4). Cross-junctions can be assembled iteratively through cycles of hybridization, crosslinking and washing. Cross-junctions can also be assembled and spatially addressed on a surface, and subsequent synthesis steps can still be performed on the surface.

Assembling a library of orthogonal cross junction arrays on a surface can enable multiplexed synthesis of orthogonal, and arbitrary length DNA of prescribed sequences.

In one exemplary embodiment, 3-Cyanovinylcarbazole (CNVK) (Vieregg et al., 2013) base modification and a UV light source to crosslink DNA junctions together is used. Strand design can utilize a set of two or more hybridization domains C1 and 1*, 2 and 2* in current implementation), an internal barcode sequence (b1, b2, etc. . . . ) that will serve as the growing sequence chain, and the CNVK base cross-linker (FIG. 4A, cyan circle).

### Nucleic Acid Synthesis

In still another aspect, the disclosure provides a method for synthesizing a nucleic acid using a nucleic acid template described herein. Generally, the method comprises extending a nucleotide sequence from the 3′-end of a primer annealed or hybridized to the nucleic acid template using a DNA polymerase having strand displacement activity.

In some embodiments, the method further comprises annealing or hybridizing a primer to the nucleic acid template. For example, the primer is annealed or otherwise hybridized to a guide strand in the nucleic acid template. In some embodiments, the primer can be annealed or hybridized to a guide strand of the nucleic acid template prior to assembling the full length nucleic acid.

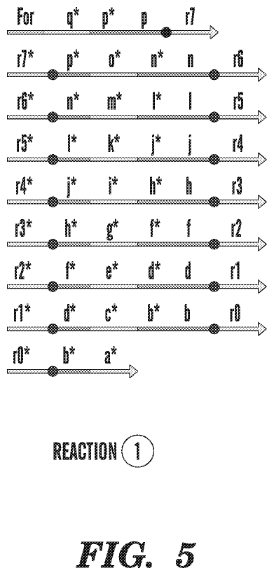

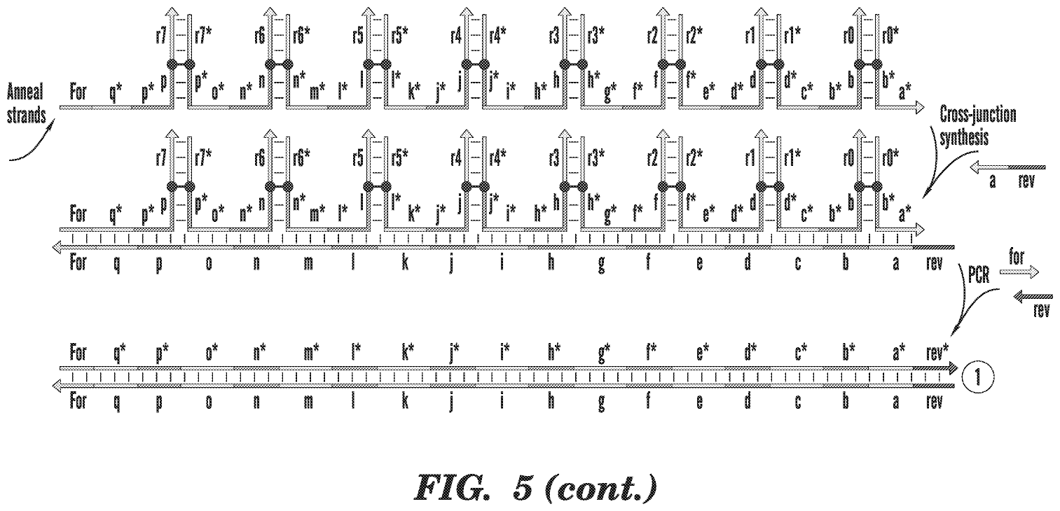

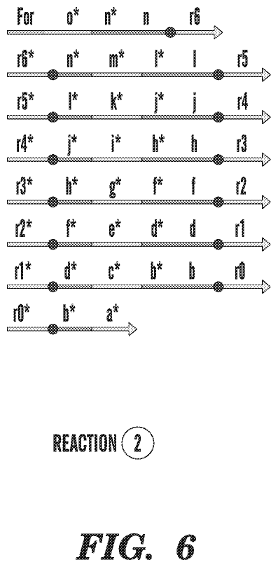

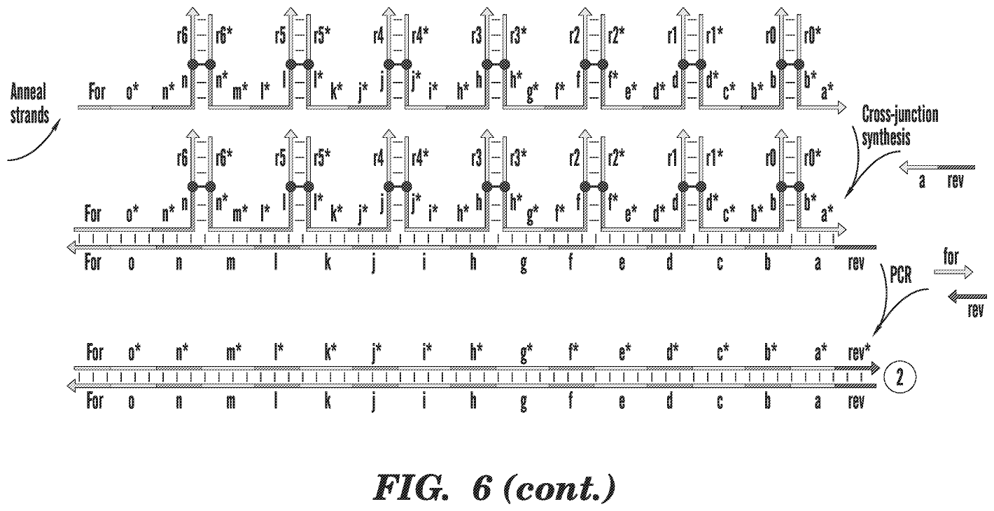

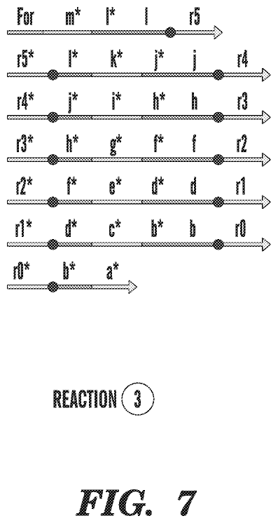

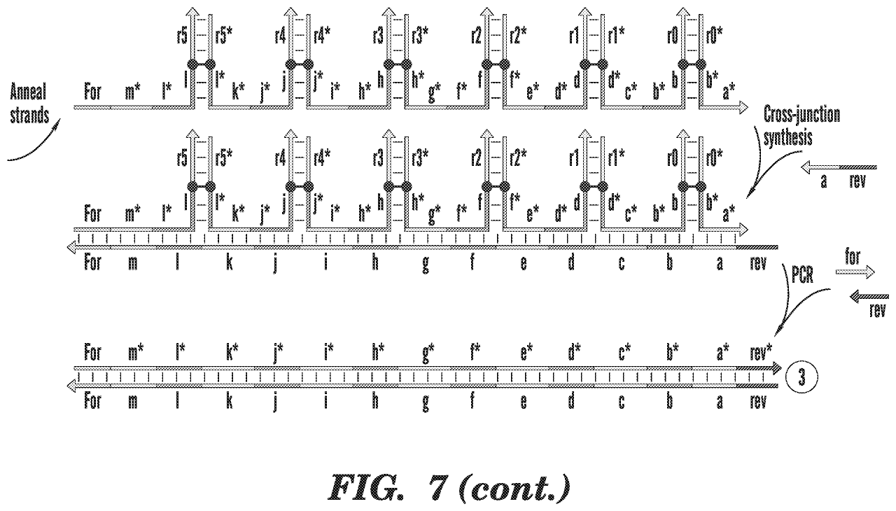

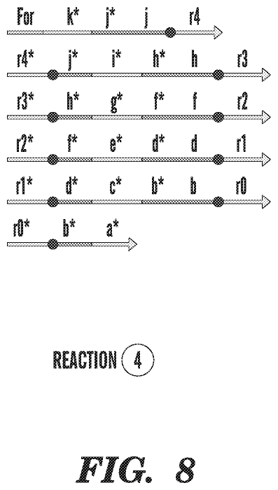

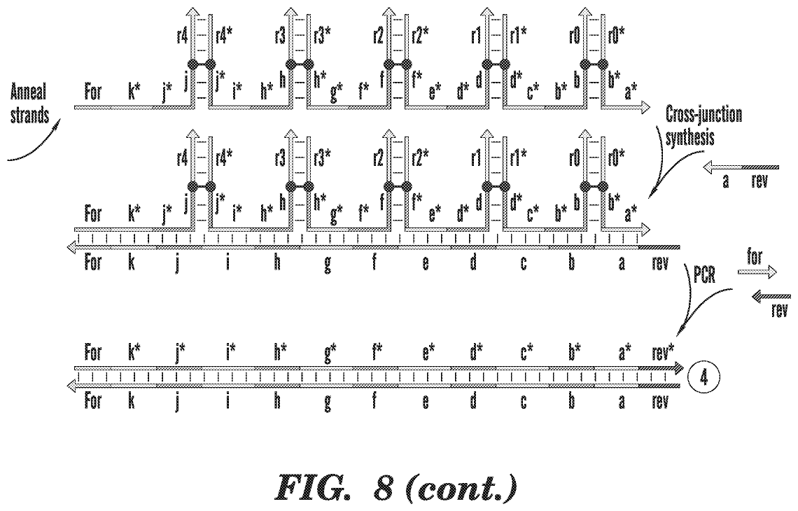

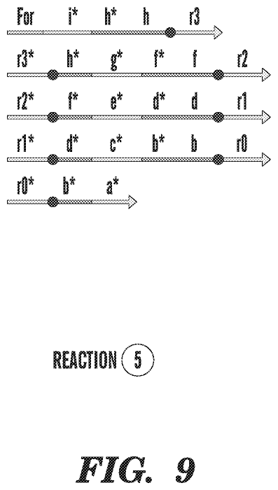

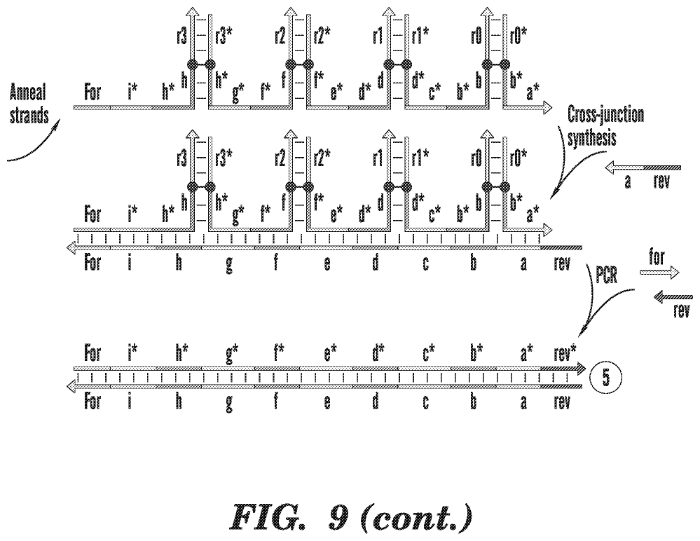

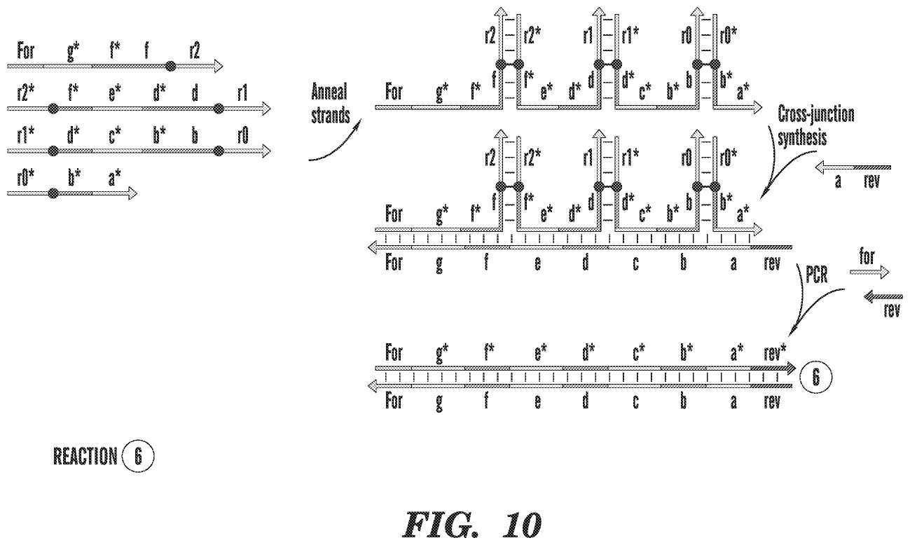

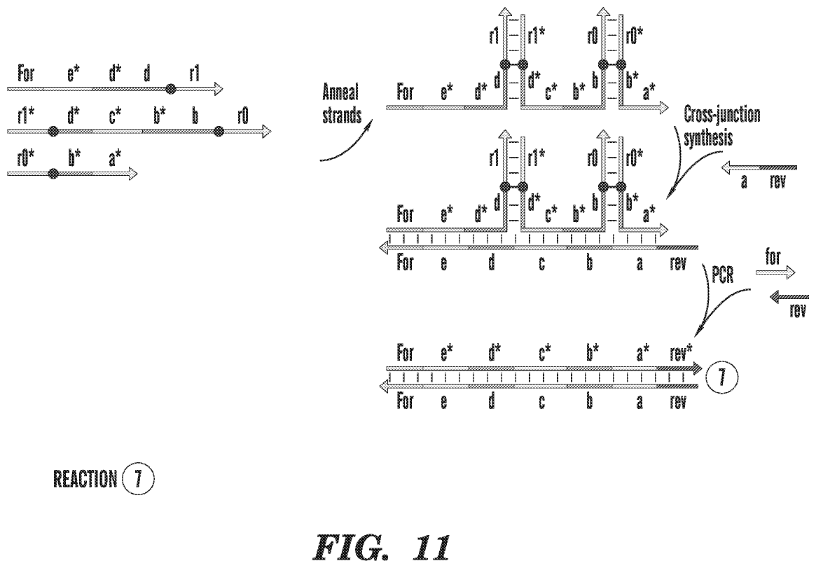

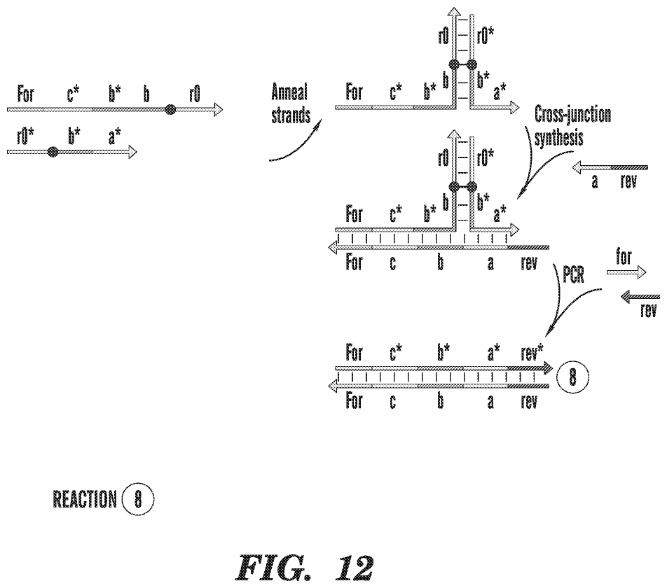

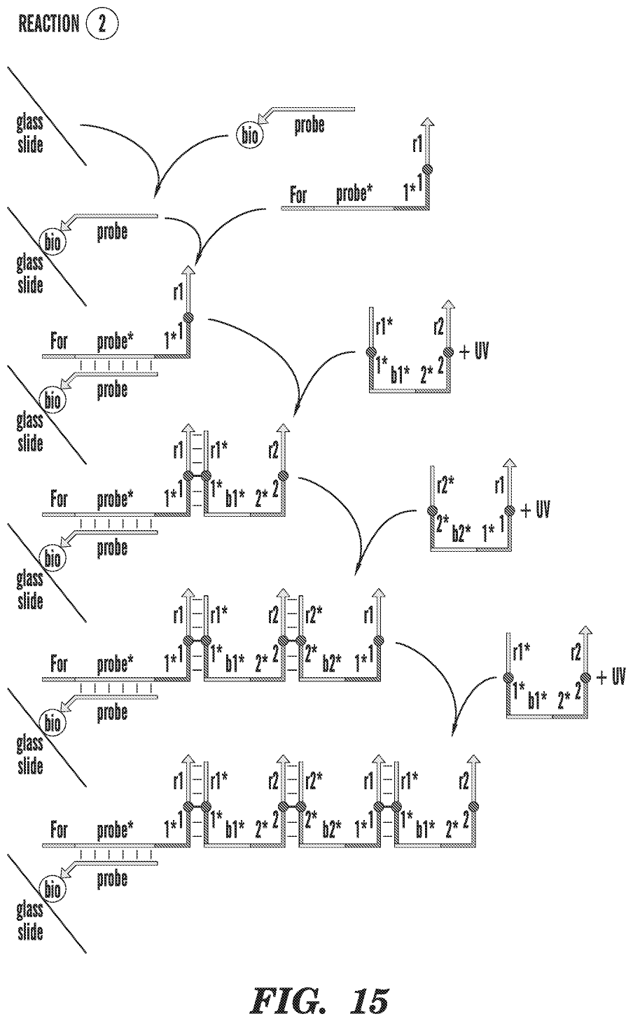

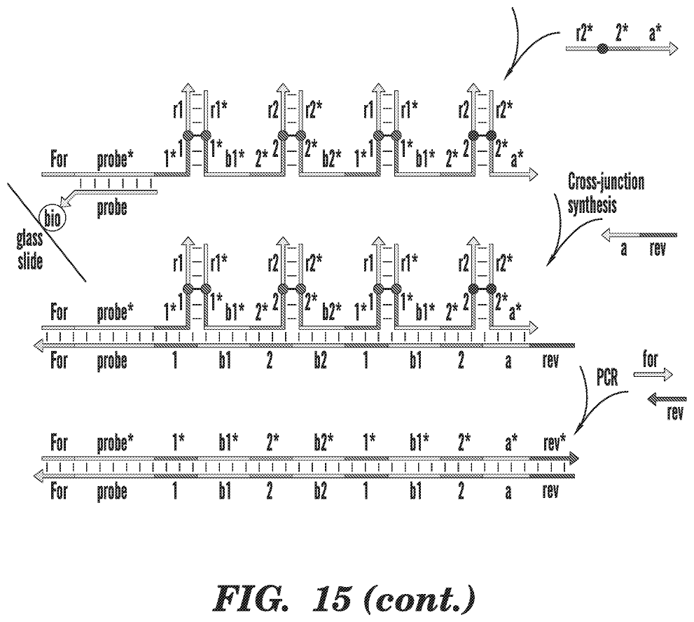

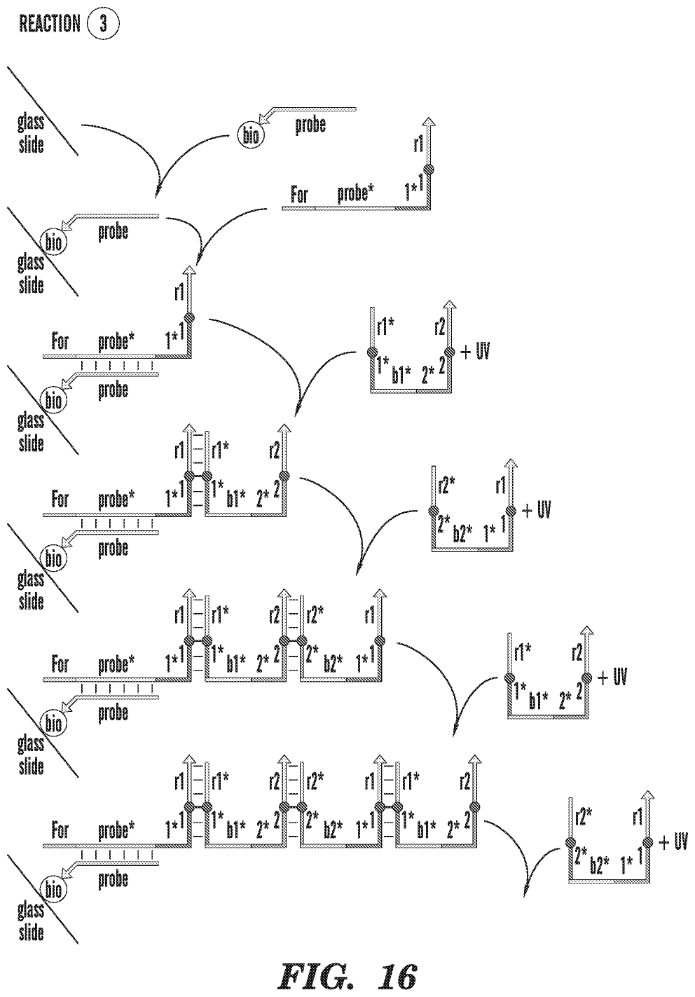

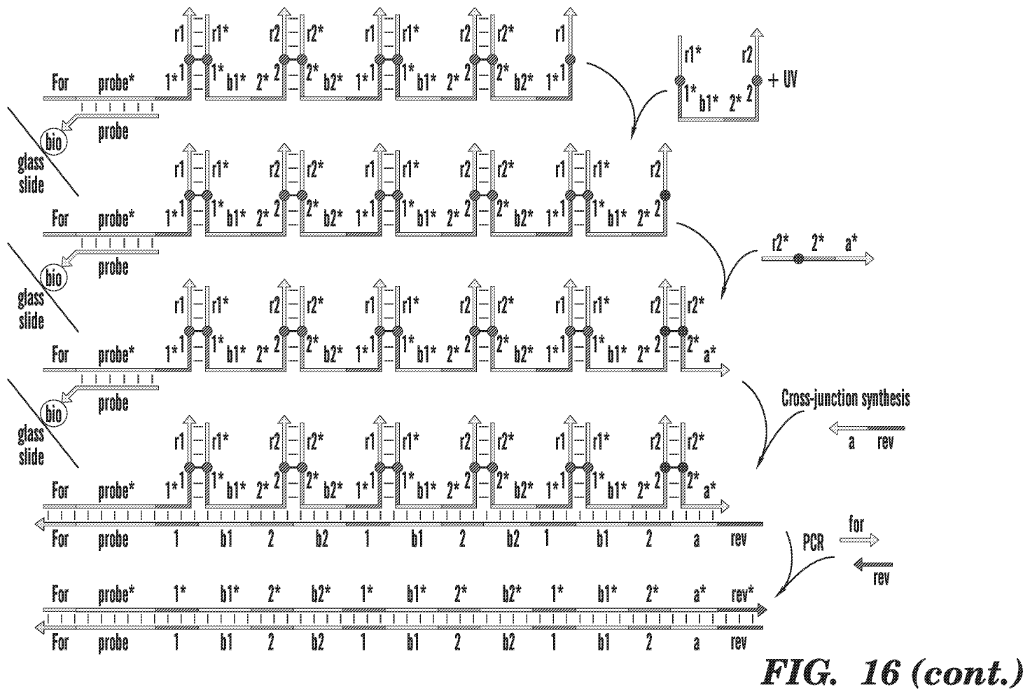

The nucleic acid template for use in the method of synthesizing a nucleic acid described herein can be fully assembled prior to the synthesis. For example, as shown in FIGS. 5-12 and 14-19A, all the desired guide strands can be annealed or hybridized together, to form the full length template, prior to extension from the primer. Thus, in some embodiments, the method for synthesizing a nucleic acid comprises annealing or hybridizing the guide strands to form the full length nucleic acid template prior to extending the nucleotide sequence from the 3′-end of the primer. For example, all the guide strands can be added in an initial reaction mixture and allowed to anneal or hybridize prior to the extension step.

Alternatively, the synthesis can be continuous. For example, the guide strands can be added consecutively to the synthesis reaction over a period of time under conditions that permit synthesis of the desired nucleic acid sequence across the junctions. Generally, the synthesis starts by extending a nucleotide sequence from the 3′-end of a primer annealed or hybridized to a guide strand. The reaction is substantially free of other guide strands of the full length template. After a period of time, e.g., 15 seconds, 30 seconds, 45 seconds, 1 minutes, 2 minutes, 3 minutes, 4 minutes, 5 minutes or more, the next guide stand is added. This can be continued until the desired full length template has been assembled.

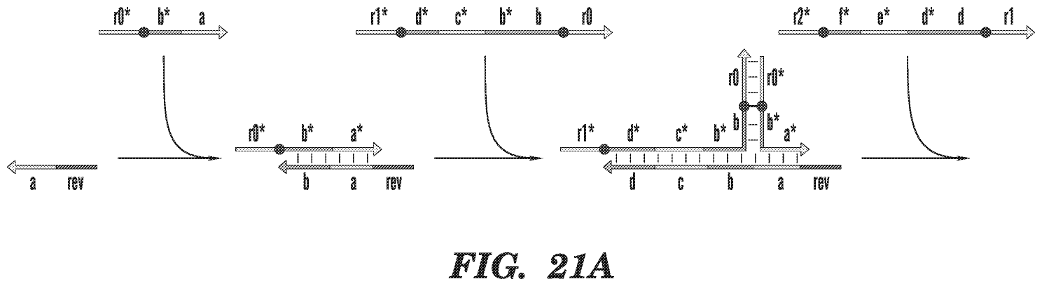

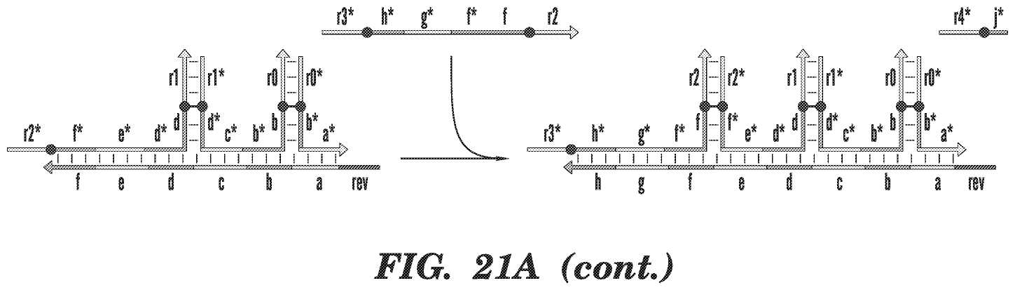

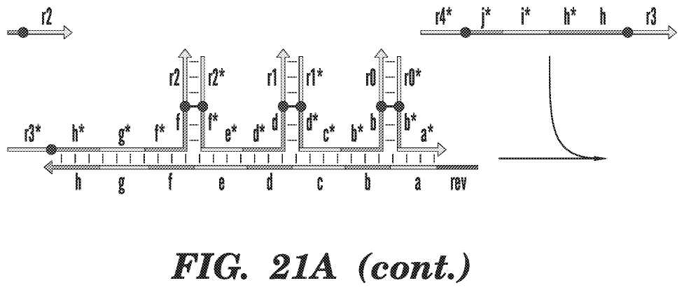

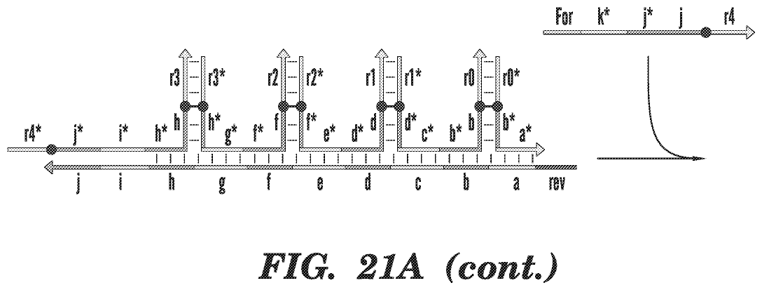

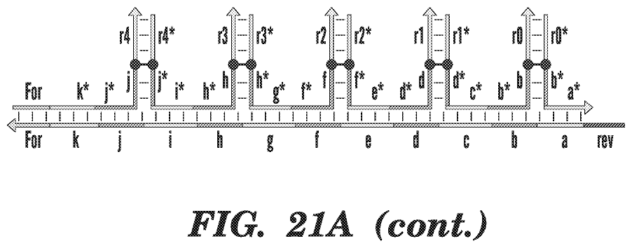

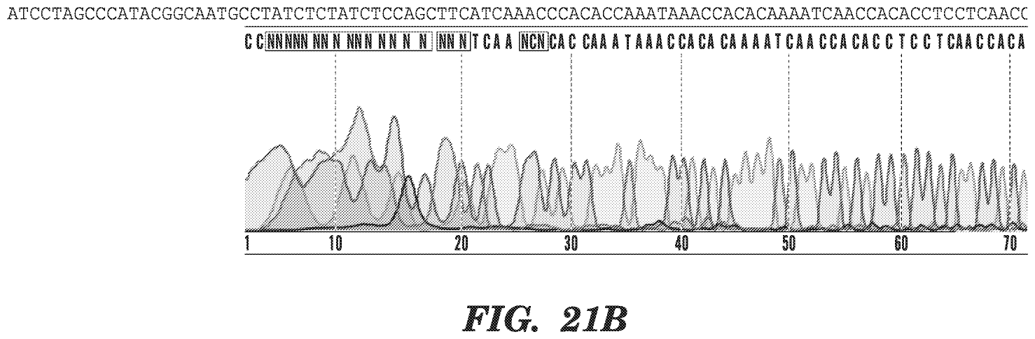

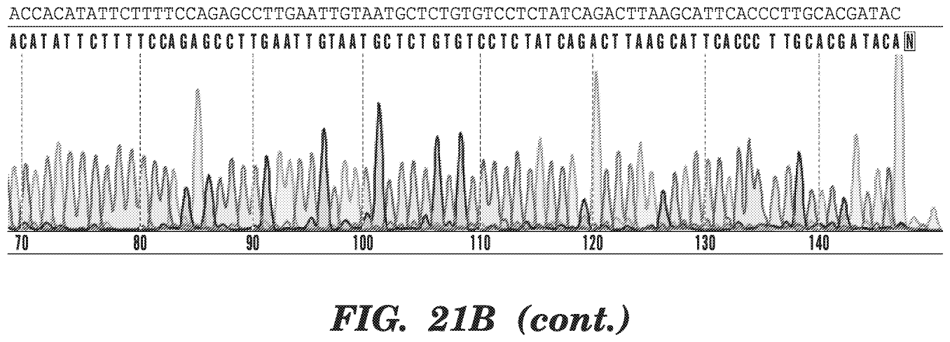

As shown in the exemplary embodiment in FIG. 21A, the synthesis starts with annealing or hybridizing a first guide strand with a primer. The first guide strand comprises in a 3′ to 5′ direction a synthesis region, a junction domain and a blocking region. After it has annealed or hybridized to the first guide strand, the primer is extended from its 3′-end. After a period of time, e.g., e.g., 5 seconds, 10 seconds, 15 seconds, 30 seconds, 45 seconds, 1 minutes, 2 minutes, 3 minutes, 4 minutes, 5 minutes, 10 minutes, 15 minutes, 20 minutes or more, additional new guide stands can be added consecutively to the synthesis reaction.

It is noted that after a new guide strand is added to the synthesis reaction, the new guide strand is allowed to anneal or otherwise hybridize to a guide strand already in the synthesis reaction. Optionally, the complementary blocking regions are covalently cross-linked, e.g., by photo-crosslinking. The synthesis is allowed to continue for a period of time, e.g., 5 seconds, 10 seconds, 15 seconds, 30 seconds, 45 seconds, 1 minutes, 2 minutes, 3 minutes, 4 minutes, 5 minutes, 10 minutes, 15 minutes, 20 minutes or more, before the next guide strand is added to the reaction.

Generally, each additional guide strand comprises in a 3′ to 5′ direction, a first blocking region, a first junction domain, a first synthesis region, a second synthesis region, a second junction domain, and a second blocking region. The first new guide strand added to the synthesis reaction has the following characteristics: the first blocking region of the new guide strand and the blocking region of the first guide strand together form a blocking domain that blocks strand displacement activity of a polymerase; the first junction domain of the new guide strand comprises a nucleotide sequence substantially complementary to a nucleotide sequence of the junction domain of the first guide strand to form a double-stranded region; and the first synthesis region of the new guide strand comprises a nucleotide sequence substantially identical to a nucleotide sequence of the junction domain of the first guide strand.

Each additional new guide strand added after the first new guide strand has the following characteristics: the first blocking region of the new guide stand and the second blocking region of the last guide strand added to the synthesis reaction together form a blocking domain that blocks strand displacement activity of a polymerase; the first junction domain of the new guide strand comprises a nucleotide sequence substantially complementary to a nucleic acid sequence of the second junction domain of the last guide strand added to the synthesis reaction to form a double-stranded region; and the first synthesis region of the new guide strand comprises a nucleotide sequence substantially identical to a nucleic acid sequence of the second junction domain of the last guide strand added to the synthesis reaction.

The number of additional guide strands that can be added is not limited. For example, 1, 2, 3, 4, 5, 6, 7, 8, 9, 10, 11, 12, 13, 14, 15, 16, 17, 18, 19, 20, 21, 22, 23, 24, 25 or more additional guide strands can be added. In some embodiments, 75, 100, 150, 200, 250 or more additional guide strands can be added.

In some embodiments, the method further comprises adding a terminal guide strand to the reaction synthesis. The terminal guide strand comprises in a 3′ to 5′ direction a blocking region, a junction domain, a first synthesis region, optionally a second synthesis region, and optionally a primer sequence.

If one or more additional guide strands were added to the synthesis reaction, the terminal guide has the following characteristics: the blocking region of the terminal guide stand and the second blocking region of the last guide strand added to the synthesis reaction together form a blocking domain that blocks strand displacement activity of a polymerase; the junction domain of the terminal guide strand comprises a nucleotide sequence substantially complementary to a nucleic acid sequence of the second junction domain of the last guide strand added to the synthesis reaction to form a double-stranded region; and the first synthesis region of the terminal guide strand comprises a nucleotide sequence substantially identical to a nucleic acid sequence of the second junction domain of the last guide strand added to the synthesis reaction.

If no additional guide strands are added to the synthesis, the terminal guide has the following characteristics: the blocking region of the terminal guide stand and the blocking region of the first guide strand together form a blocking domain that blocks strand displacement activity of a polymerase; the junction domain of the terminal guide strand comprises a nucleotide sequence substantially complementary to a nucleic acid sequence of the junction domain of the first guide strand to form a double-stranded region; and the first synthesis region of the terminal guide strand comprises a nucleotide sequence substantially identical to a nucleic acid sequence of the junction domain of the first guide strand added to the synthesis reaction.

In some embodiments, the terminal guide strand comprises a primer sequence at its 5′-end.

As used herein, the term “primer” is used to describe a sequence of DNA (or RNA) that is paired with one strand of DNA and provides a free 3′-OH at which a DNA polymerase starts synthesis of a deoxyribonucleotide chain. Preferably, the primer is composed of an oligonucleotide. The exact lengths of the primers will depend on many factors, including temperature and source of primer. For example, depending on the complexity of the target sequence, the oligonucleotide primer typically contains 15-25 or more nucleotides, although it may contain fewer nucleotides. Short primer molecules generally require cooler temperatures to form sufficiently stable hybrid complexes with template.

A “polymerase” refers to an enzyme that performs template-directed synthesis of polynucleotides, e.g., DNA and/or RNA. The term encompasses both the full length polypeptide and a domain that has polymerase activity. DNA polymerases are well-known to those skilled in the art, including but not limited to DNA polymerases isolated or derived from Pyrococcus furiosus, Thermococcus litoralis, and Thermotoga maritime, or modified versions thereof. Additional examples of commercially available polymerase enzymes include, but are not limited to: Klenow fragment (New England Biolabs® Inc.), Taq DNA polymerase (QIAGEN), 9° N™ DNA polymerase (New England Biolabs® Inc.), Deep Vent™ DNA polymerase (New England Biolabs® Inc.), Manta DNA polymerase (Enzymatics®), Bst DNA polymerase (New England Biolabs® Inc.), and phi29 DNA polymerase (New England Biolabs® Inc.). Polymerases include both DNA-dependent polymerases and RNA-dependent polymerases such as reverse transcriptase. At least five families of DNA-dependent DNA polymerases are known, although most fall into families A, B and C. There is little or no sequence similarity among the various families. Most family A polymerases are single chain proteins that can contain multiple enzymatic functions including polymerase, 3′ to 5′ exonuclease activity and 5′ to 3′ exonuclease activity. Family B polymerases typically have a single catalytic domain with polymerase and 3′ to 5′ exonuclease activity, as well as accessory factors. Family C polymerases are typically multi-subunit proteins with polymerizing and 3′ to 5′ exonuclease activity. In E. coli, three types of DNA polymerases have been found, DNA polymerases I (family A), II (family B), and III (family C). In eukaryotic cells, three different family B polymerases, DNA polymerases α, δ, and ε, are implicated in nuclear replication, and a family A polymerase, polymerase γ, is used for mitochondrial DNA replication. Other types of DNA polymerases include phage polymerases. Similarly, RNA polymerases typically include eukaryotic RNA polymerases I, II, and III, and bacterial RNA polymerases as well as phage and viral polymerases. RNA polymerases can be DNA-dependent and RNA-dependent.

It is noted that reagents, such as strand displacing DNA or RNA polymerases, and methods for synthesizing nucleic acid sequences from nucleic acid templates are well known in the art and are amenable to the invention. See, for example, US20050277146A1, US20100035303A1, and WO2006030455A1, contents of all of which are incorporated herein by reference in their entirety.

In some embodiments of any of the aspects, the method further comprises amplifying the nucleic acid sequence. As used herein, the term “amplifying” refers to a step of submitting a nucleic acid sequence to conditions sufficient to allow for amplification of a polynucleotide if all of the components of the reaction are intact. Components of an amplification reaction include, e.g., primers, a polynucleotide template, polymerase, nucleotides, and the like. The term “amplifying” typically refers to an “exponential” increase in target nucleic acid. However, “amplifying” as used herein can also refer to linear increases in the numbers of a select target sequence of nucleic acid, such as is obtained with cycle sequencing. Methods of amplifying and synthesizing nucleic acid sequences are known in the art. For example, see U.S. Pat. Nos. 7,906.282, 8,367,328, 5,518,900, 7,378,262, 5,476,774, and 6,638,722, contents of all of which are incorporated by reference herein in their entirety.

In some embodiments, amplifying the nucleic acid sequence comprises a polymerase chain reaction (PCR). PCR is well known to those of skill in the art; see, e.g., U.S. Pat. Nos. 4,683,195 and 4,683,202; and PCR Protocols: A Guide to Methods and Applications, Innis et al., eds, 1990, contents of all which are incorporated herein by reference in their entirety. Exemplary PCR reaction conditions typically comprise either two or three step cycles. Two step cycles have a denaturation step followed by a hybridization/elongation step. Three step cycles comprise a denaturation step followed by a hybridization step followed by a separate elongation step.

In some embodiments, the amplification step includes additional polynucleotide sequences or templates with hairpins that are orthogonal the amplification step. Without wishing to be bound by a theory, such additional DNA hairpins can reduce or correct for off-target reactions. For example, when a three-letter code is used, these additional hairpin comprising sequences or templates can serve to soak up the trace amounts of unwanted nucleotide that can be present in some samples.

In some embodiments of any of the aspects described herein, the guide strands are added in an initial reaction mixture. In some embodiments of any of the aspects described herein, the synthesis is continuous. In continuous synthesis, the guide strands are each added consecutively under conditions that permit synthesis of the desired nucleic acid across the junctions. See for example, FIG. 21A in the working examples, which provides a schematic representation of one embodiment of the methods described herein.

To complete a round of continuous nucleic acid synthesis as described herein, a terminal barcode strand can be used. As used herein, a “terminal barcode strand” comprises (i) one or more substantially identical binding domains to one template strand; and optionally, (ii) a different barcode; and optionally (iii) a PCR primer sequence on the 5′ end to enable complete synthesis products to be exponentially amplified. Specifically, the optional barcode of the terminal barcode strand is not complementary to the template strand. Without limitations, the terminal barcode strand can be added to the 3′ or 5′ end of the template nucleic acid. The terminal barcode strand can further comprise a label or a fluorophore.

In another aspect, provided herein is a reaction mixture comprising a nucleic acid template described.

In some embodiments of any of the aspects, the nucleic acid template further comprises a primer annealed or otherwise hybridized to the one of the guide strands. In another embodiment, the nucleic acid template further comprises a primer annealed or hybridized to the first guide strand, second guide strand, third guide strand, fourth guide strand, fifth guide strand, sixth guide strand, seventh guide strand, etc.

In some embodiments of any of the aspects, the reaction mixture further comprises nucleotide triphosphates or deoxynucleotide triphosphates. In some embodiments, the reaction mixture does not include one of adenosine, thymidine/uridine, cytosine or guanosine triphosphate or deoxynucleotide triphosphate.

In some embodiments, the reaction mixture further comprises a DNA or RNA polymerase.

In some embodiments, the reaction mixture further comprises a buffer or salt for nucleic acid synthesis. It is contemplated that buffer used in the reaction mixture is chosen that permit the stability of the nucleic acid template and desired nucleic acid sequence. Methods of choosing such buffers are known in the art and can also be chosen for their properties in various conditions including pH or temperature of the reaction being performed.

In some embodiments, the reaction mixture comprises components that can be utilized to create the guide strands by addition of junction regions onto existing nucleic acid strands enzymatically or chemically. For example, a reaction mixture that can be utilized to create the guide strands by addition of blocking regions onto existing nucleic acid strands enzymatically or chemically.

In one aspect, provided herein is a kit comprising the nucleic acid template as provided herein. In certain embodiments, such kits are intended for therapeutic application. In certain embodiments, such kits are intended for research use.

In one embodiment of any of the aspects, the kit further comprises nucleotide triphosphates or deoxynucleotide triphosphates.

In another embodiment of any of the aspects, the kit further comprises a DNA or RNA polymerase.

In one embodiment of any of the aspects, the kit further comprises a buffer or salt for nucleic acid synthesis.

In one embodiment of any of the aspects, the kit further comprises solid supports as provided herein to isolate specific sequences.

In yet another aspect, provided herein is a kit comprising components or reaction mixture for creating the nucleic acid template described herein from nucleic acid sequences (partial guide strands) by addition of junction regions and/or blocking regions onto existing nucleic acid strands enzymatically or chemically.

In some embodiments of the various aspects described herein, the kit further comprises instructions for use.

Methods of purification and analysis of nucleic acids are known to those skilled in the art. Non-limiting examples of methods to characterize the nucleic acid synthesis include liquid chromatography, mass spectrometry, next generation sequencing, polymerase chain reaction (PCR), gel electrophoresis, or any other method of identifying nucleoside sequences, secondary structures, chemical composition, expression, thermodynamics, binding, or function.

### Exemplary Applications

The nucleic acid template and methods provided herein are fundamentally enabling for several different applications. Non-limiting examples include, but are not limited to, those described below.

Proximity and molecular distance measurements. Because the synthesis step happens isothermally, and the hybridization kinetics can be tuned to also happen under mild (non-denaturing) conditions, the proximity of biomolecules can be determined and create records indicative of distance information between labeled biomolecules. This has applications in distance/proximity measurements of DNA/RNA/proteins and other biomolecules of interest, structure determination of DNA, RNA, protein, and RNA-protein complexes. This could be further used as a method for fingerprinting of proteins and other biomolecules, if the synthesis reactions are programmed to produce relatively distinct record patterns from other targets.

Long strand synthesis and DNA assembly. Because the assembly and synthesis steps can be decoupled, assembly of larger sequences than is possible with other methods may be performed.

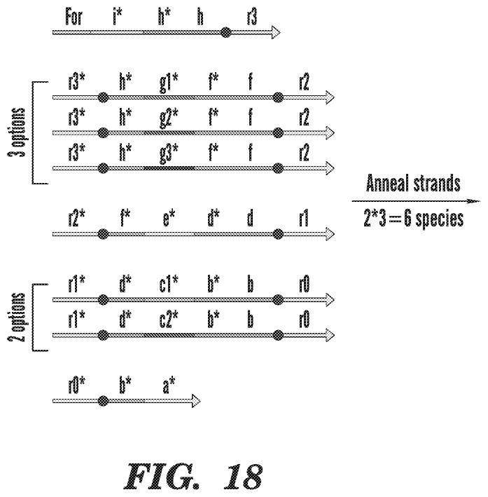

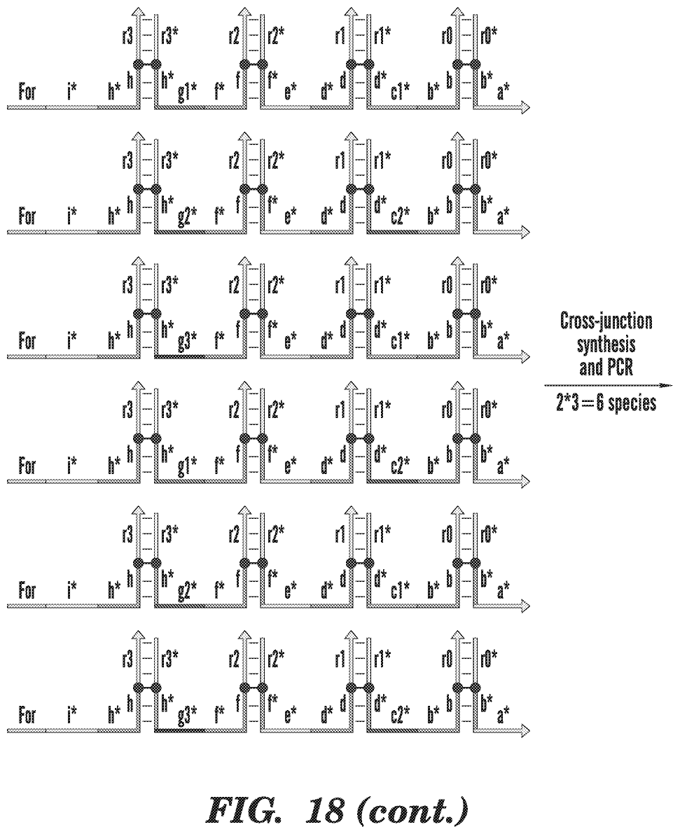

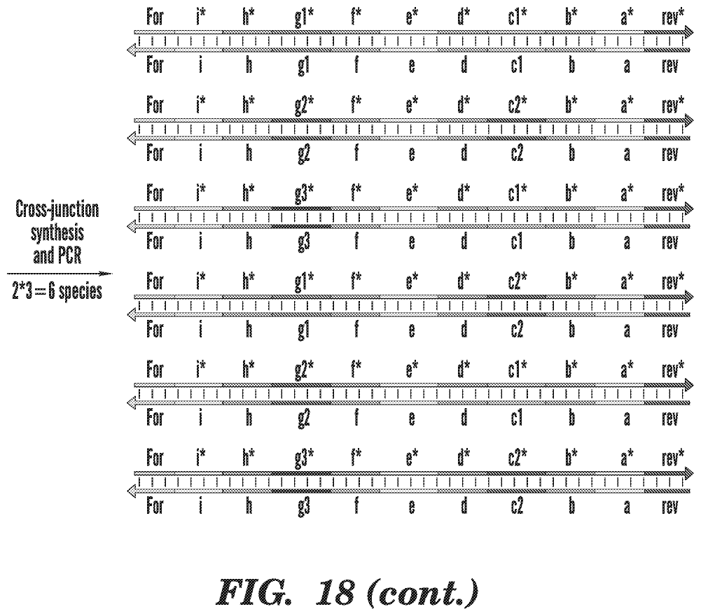

Combinatorial synthesis for library generation. The template concatemerization can be designed to produce a programmable combinatorial set of sequences (FIG. 18). Multiple options of intercalating domains may be desired in a sequence library, and this can be implemented by simply including the relevant guide strands with each of the options included at the desired relative frequency. For example, 10 options for each of 5 different domains would require 10*5=50 guide strands in place of the usual 5 for fixed domains, but would enable the creation of 10\hat{ }5=100,000 possible sequences produced. In general, for positions p1, p2, . . . , pn, each with a number of possibilities x1, x3, . . . , xn, the total number of strands required for those positions would be x1+x2+ . . . +xn, and they can produce a library of size x1*x3* . . . * xn.

Parallel synthesis of multiple orthogonal sequences. Multiple orthogonal cross-junction synthesis reaction can be performed together in the same reaction solution. These may or may not use the same primer or intercalating sequences.

In-situ synthesis. The synthesis step is performed in an aqueous, isothermal environment, allowing in-situ synthesis of arbitrary programmable strands within biological samples or biological materials.

Constructing identity barcodes (unique molecular identifiers, batch numbers, indices). The cross junction synthesis can be used to create unique identity barcodes in a combinatorial manner on surfaces, matrices, biomolecules, molecular libraries or biological samples or materials (e.g., vesicles, cells, tissues, organoids, droplets, liposomes, small molecules, beads) or to encode unique molecular identities to spatial positions, biomolecules or biological samples or materials. This can be further combined with early approaches such as split-and-pool synthesis or split-pool synthesis where addition of each guide strand is performed following the split of the target population and prior to pooling. See for example, Brenner et al. (2000) Proc. Natl. Acad. Sci. USA 97:1665 and US20160251697A1, which are incorporated herein by reference in their entirety.

Constructing spatial barcodes. The cross-junction synthesis can be used to create barcodes in a combinatorial manner at specified positions or specified targets on surfaces. Non-limiting examples of target on surfaces include biomolecules, biological materials, and the like. The biological material can be selected from tissues, tissue sections, engineered tissues, cells, patient derived cells, primary cells, organoids, extracellular matrix, 3D biological organs, dissociated cells, live cells, fixed cells, vesicles, droplets, liposomes, etc.

Strand re-routing. Because cross-junction synthesis can be used to ‘re-route’ a polymerase to a new backbone sequence, it may be used for gene and sequence editing with synthesis re-routing. This can be done by binding a strand with the new desired sequence in front of the old sequence so that the polymerase copies across a junction onto the new sequence.

In addition to the applications provided above, several types of nucleic acid synthesis can be employed using the compositions and methods provided herein. Synthesis can include but is not limited to single-stranded DNA synthesis, double-stranded DNA synthesis, mutagenesis, insertions, deletions, homologous recombination, parallel/multiplexed synthesis, proximity and molecular distance measurements.

### Selected Definitions

For convenience, the meaning of some terms and phrases used in the specification, examples, and appended claims, are provided below. Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments of the aspects provided herein, and are not intended to limit the claimed invention, because the scope of the invention is limited only by the claims. Further, unless otherwise required by context, singular terms shall include pluralities and plural terms shall include the singular.

Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

As used herein, “nucleic acid” means DNA, RNA, single-stranded, double-stranded, or more highly aggregated hybridization motifs, and any chemical modifications thereof. For example, a nucleic acid can encompass double- or triple-stranded nucleic acids, as well as single-stranded molecules. In double- or triple-stranded nucleic acids, the nucleic acid strands need not be coextensive (i.e., a double-stranded nucleic acid need not be double-stranded along the entire length of both strands). The term nucleic acid also encompasses any chemical modification thereof, such as by methylation and/or by capping. Nucleic acid modifications can include addition of chemical groups that incorporate additional charge, polarizability, hydrogen bonding, electrostatic interaction, and functionality to the individual nucleic acid bases or to the nucleic acid as a whole. Such modifications may include base modifications such as 2′-position sugar modifications, 5-position pyrimidine modifications, 8-position purine modifications, modifications at cytosine exocyclic amines, substitutions of 5-bromo-uracil, backbone modifications, unusual base pairing combinations such as the isobases isocytidine and isoguanidine, and the like. The nucleic acid(s) can be derived from a completely chemical synthesis process, such as a solid phase-mediated chemical synthesis, from a biological source, such as through isolation from any species that produces nucleic acid, or from processes that involve the manipulation of nucleic acids by molecular biology tools, such as DNA replication, PCR amplification, reverse transcription, or from a combination of those processes.

As used herein, the terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three dimensional structure, and may perform any function, known or unknown. The following are non-limiting examples of polynucleotides: coding or non-coding regions of a gene or gene fragment, intergenic DNA, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), small nucleolar RNA, ribozymes, complementary DNA (cDNA), which is a DNA representation of mRNA, usually obtained by reverse transcription of messenger RNA (mRNA) or by amplification; DNA molecules produced synthetically or by amplification, genomic DNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “statistically significant” or “significantly” refers to statistical significance and generally means a two standard deviation (2SD) or greater difference.

As used herein the term “comprising” or “comprises” is used in reference to compositions, methods, and respective component(s) thereof, that are essential to the method or composition, yet open to the inclusion of unspecified elements, whether essential or not.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those provided herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Further, unless otherwise required by context, singular terms shall include pluralities and plural terms shall include the singular.

Other than in the operating examples, or where otherwise indicated, all numbers expressing quantities of ingredients or reaction conditions used herein should be understood as modified in all instances by the term “about.” The term “about” when used in connection with percentages can mean ±1%.

The term “substantially identical” means two or more nucleotide sequences have at least 65%, 70%, 80%, 85%, 90%, 95%, or 97% identical nucleotides. In some embodiments, “substantially identical” means two or more nucleotide sequences have the same identical nucleotides.

As used herein, and unless otherwise indicated, the term “complementary,” when used to describe a first nucleotide sequence in relation to a second nucleotide sequence, refers to the ability of an oligonucleotide or polynucleotide comprising the first nucleotide sequence to hybridize and form a duplex structure under certain conditions with an oligonucleotide or polynucleotide comprising the second nucleotide sequence, as will be understood by the skilled person. Such conditions can, for example, be stringent conditions, where stringent conditions may include: 400 mM NaCl, 40 mM PIPES pH 6.4, 1 mM EDTA, 50° C. or 70° C. for 12-16 hours followed by washing. Other conditions, such as physiologically relevant conditions as may be encountered inside an organism, can apply. The skilled person will be able to determine the set of conditions most appropriate for a test of complementarity of two sequences in accordance with the ultimate application of the hybridized nucleotides.

“Complementary” sequences, as used herein, may also include, or be formed entirely from, non-Watson-Crick base pairs and/or base pairs formed from non-natural and modified nucleotides, in as far as the above requirements with respect to their ability to hybridize are fulfilled. Such non-Watson-Crick base pairs includes, but not limited to, G:U Wobble or Hoogsteen base pairing.

As used herein, the terms “substrate” or “substrate surface” are used interchangeably to describe a structure upon which one or more nucleic acid templates, guide strands, or portions thereof as provided herein can be displayed or in contact with for contact with additional nucleic acids and/or labels. The nucleic acid template, guide strand, and barcode strands provided herein can be immobilized, conjugated to, or linked to the substrate surface.

As used herein, the term “conjugated to” encompasses association of a nucleic acid with a substrate surface, a phase-changing agent or a member of an affinity pair by covalent bonding, including but not limited to cross-linking via a cross-linking agent, or by a strong non-covalent interaction that is maintained under conditions in which the conjugate is to be used.

As used herein, the term “hybridize” refers to the phenomenon of a single-stranded nucleic acid or region thereof forming hydrogen-bonded base pair interactions with either another single stranded nucleic acid or region thereof (intermolecular hybridization) or with another single-stranded region of the same nucleic acid (intramolecular hybridization). Hybridization is governed by the base sequences involved, with complementary nucleobases forming hydrogen bonds, and the stability of any hybrid being determined by the identity of the base pairs (e.g., G:C base pairs being stronger than A:T base pairs) and the number of contiguous base pairs, with longer stretches of complementary bases forming more stable hybrids.

As used herein, the term “spatial information” is any information, coordinates, markers in a biological tissue or matrix, that can be stored in the barcode. The spatial information can inform one of skill in the art where on the substrate a particular marker, barcode, or pattern is located. For example, spatial information may be useful in creating an image or QR code with the nucleic acid barcodes. Spatial information can also be useful in the detection of a specific nucleic acid target.

Some embodiments of the methods and compositions described herein can be defined according to any of the following numbered paragraphs:

1) A nucleic acid template comprising:
(a) a first guide strand comprising in 3′ to 5′ direction a first synthesis region, a second synthesis region, a first junction domain, and a first blocking region; and
(b) a second guide strand comprising in 3′ to 5′ direction a second blocking region, a second junction domain and a third synthesis region, wherein the first junction domain comprises a nucleotide sequence substantially identical to a nucleotide sequence of the third synthesis region, and the first junction domain and second junction domain are substantially complementary to each other and form a double-stranded region, and wherein the first blocking region and the second blocking region together form a first blocking domain that blocks strand displacement activity of a polymerase.

2) The nucleic acid template of paragraph 1, wherein the first and the second blocking regions are covalently linked to each other.

3) The nucleic acid template of any one of paragraphs 1-2, wherein the first or the second blocking region comprises a cross-linking segment with the other blocking region to form the first blocking domain.

4) The nucleic acid template of paragraph 3, wherein the cross-linking segment comprises 3-cyanovinylcarbozole.

5) The nucleic acid template of any one of paragraphs 1-4, wherein the second guide strand further comprises a fourth synthesis region at the 5′ end of the third synthesis region.

6) The nucleic acid template of any one of paragraphs 1-5, wherein the second guide strand further comprises at its 5′end in 3′ to 5′ direction a third junction domain and a third blocking region, and the nucleic acid template further comprises a third guide strand comprising in 3′ to 5′ direction a fourth blocking region, a fourth junction domain and a fifth synthesis region,
wherein the third junction domain comprises a nucleotide sequence substantially identical to a nucleotide sequence of the fifth synthesis region,
wherein the third junction domain and fourth junction domain are substantially complementary to each other and form a double-stranded region, and
wherein the third blocking region and the fourth blocking region together form a second blocking domain that blocks strand displacement activity of a polymerase.

7) The nucleic acid template of any one of paragraphs 1-6, wherein the third and the fourth blocking regions are covalently linked to each other.

8) The nucleic acid template of any one of paragraphs 1-7, wherein the third or the fourth blocking region comprises a cross-linking segment with the other blocking region to form the second blocking domain.

9) The nucleic acid template of paragraph 8, wherein the cross-linking segment comprises 3-cyanovinylcarbozole.

10) The nucleic acid template of any one of paragraphs 6-9, wherein the third guide strand further comprises a sixth synthesis region at the 5′ end of the fifth synthesis region.

11) The nucleic acid template of any one of paragraphs 6-10, wherein the third guide strand further comprises at its 5′ end in 3′ to 5′ direction a fifth junction domain and a fifth blocking region, and the nucleic acid template further comprises a fourth guide strand comprising in 3′ to 5′ direction a sixth blocking region, a sixth junction domain and a seventh synthesis region,
wherein the fifth junction domain comprises a nucleotide sequence substantially identical to a nucleotide sequence of the seventh synthesis region, and the fourth junction domain and fifth junction domain are substantially complementary to each other and form a double-stranded region, and
wherein the fifth blocking region and the sixth blocking region together form a third blocking domain that blocks strand displacement activity of a polymerase.

12) The nucleic acid template of paragraph 11, wherein the fifth and sixth blocking domains are covalently linked to each other.

13) The nucleic acid template of paragraphs 11 or 12, wherein the fifth or the sixth blocking region comprises a cross-linking segment with the other blocking region to form the third blocking domain.

14) The nucleic acid template of paragraph 13, wherein the cross-linking segment comprises 3-cyanovinylcarbozole.

15) The nucleic acid template of any one of paragraphs 11-14, wherein the fourth guide strand further comprises an eighth synthesis region at the 5′ end of the sixth synthesis region.

16) The nucleic acid template of any one of paragraphs 11-15, wherein the fourth guide strand further comprises at its 5′end in 3′ to 5′ direction a seventh junction domain and a seventh blocking region, and the nucleic acid template further comprises a fifth guide strand comprising in 3′ to 5′ direction an eighth blocking region, an eighth junction domain and a ninth synthesis region,
wherein the seventh junction domain comprises a nucleotide sequence substantially identical to a nucleotide sequence of the ninth synthesis region, and the seventh junction domain and eighth junction domain are substantially complementary to each other and form a double-stranded region, and
wherein the seventh blocking region and the eighth blocking region together form a fourth blocking domain that blocks strand displacement activity of a polymerase

17) The nucleic acid template of claim 16, wherein the seventh and eighth blocking domains are covalently linked to each other.

18) The nucleic acid template of paragraphs 16 or 17, wherein the seventh or the eighth blocking region comprises a cross-linking segment with the other blocking region to form the fourth blocking domain.

19) The nucleic acid template of paragraph 18, wherein the cross-linking segment comprises 3-cyanovinylcarbozole.

20) The nucleic acid template of any one of paragraphs 1-19, wherein one or more regions utilize a 3-letter code.

21) The nucleic acid template of any one of paragraphs 1-20, wherein at least one of the first, second, third or fourth blocking domain comprises a double stranded region.

22) The nucleic acid template of any one of paragraphs 1-21, wherein the nucleic acid template comprises a nucleic acid modification.

23) The nucleic acid template of any one of paragraphs 1-22, wherein at least one of the first, second, third or fourth blocking domain comprises a nucleic acid modification.

24) The nucleic acid template of paragraphs 22 or 23, wherein the nucleic acid modification is a modified nucleobase.

25) The nucleic acid template of any one of paragraphs 1-24, wherein at least one of the first, second, third or fourth blocking domain comprises a poly monomer stretch.

26) The nucleic acid template of any one of paragraphs 1-25, wherein the nucleic acid template is conjugated to a solid support.

27) The nucleic acid template of any one of paragraphs 1-26, wherein the nucleic acid template further comprises a primer annealed or hybridized to the one of the guide strands.

28) The nucleic acid template of any one of paragraphs 1-27, wherein the nucleic acid template further comprises a primer annealed or hybridized to the first guide strand.

29) A method for forming a nucleic acid template, the method comprising annealing or hybridizing a first guide strand and a second guide strand, wherein:
(a) the first guide strand comprises in 3′ to 5′ direction a first synthesis region, a second synthesis region, a first junction domain, and a first blocking region; and
(b) the second guide strand comprises in 3′ to 5′ direction a second blocking region, a second junction domain and a third synthesis region,
wherein the first junction domain comprises a nucleotide sequence substantially identical to a nucleotide sequence of the third synthesis region, and the first junction domain and second junction domain are substantially complementary to each other and form a double-stranded region, and
wherein the first blocking region and the second blocking region together form a blocking domain that blocks strand displacement activity of a polymerase.

30) The method of paragraph 29, wherein the first or the second blocking region comprises a cross-linking segment with the other region to form the blocking domain and the method further comprises forming the cross-link.

31) A reaction mixture comprising a nucleic acid template of any one of paragraphs 1-28.

32) The reaction mixture of paragraph 31, further comprising nucleotide triphosphates or deoxynucleotide triphosphates.

33) The reaction mixture of paragraph 32, wherein the reaction mixture does not include one of adenosine, thymidine/uridine, cytosine or guanosine triphosphate or deoxynucleotide triphosphate.

34) The reaction mixture of any one of paragraphs 31-33, further comprising a DNA or RNA polymerase.

35) The reaction mixture of any one of paragraphs 31-35, further comprising a buffer or salt for nucleic acid synthesis.

36) A reaction mixture for synthesizing a guide strand by chemical or enzymatic addition of a junction domain to a nucleic acid.

37) A reaction mixture for synthesizing a guide strand by chemical or enzymatic addition of a blocking region to a nucleic acid stand.

38) A kit comprising the nucleic acid template of any one of paragraphs 1-28.

39) A kit comprising the components or reaction mixture for creating the nucleic acid template of any one of paragraphs 1-28 from nucleic acid sequences (partial guide strands) by enzymatic or chemical addition of junction regions and/or blocking regions onto existing nucleic acid strands.

40) The kit of paragraphs 38 or 39, further comprising nucleotide triphosphates or deoxynucleotide triphosphates.

41) The kit of claim any one of paragraphs 38-40, further comprising a DNA or RNA polymerase.

42) The kit of any one of paragraphs 38-41, further comprising a buffer or salt for nucleic acid synthesis.

43) A method for synthesizing a nucleic acid sequence, the method comprising:
(a) providing or obtaining a nucleic acid template of any one of paragraphs 1-28;
(b) annealing or hybridizing a primer, if not already annealed, to the nucleic acid template; and
(c) extending a nucleotide sequence using a DNA polymerase having strand displacement activity from the 3′-terminus of the primer.

44) The method of paragraph 43, further comprising amplifying the nucleic acid sequence.

45) A method for synthesizing a nucleic acid sequence, the method comprising:

(a) annealing or hybridizing a primer, if not already annealed, to a first guide strand, wherein the first guide strand comprises in a 3′ to 5′ direction a first synthesis region, a second synthesis region, a junction domain and a blocking region; and
(b) extending a nucleotide sequence using a DNA polymerase having strand displacement activity from the 3′-terminus of the primer;
(c) adding a second guide strand to the synthesis reaction, where the second guide strand comprises in a 3′ to 5′ direction a blocking region, a junction domain, a first synthesis region, a second synthesis region, a second junction domain, and a second blocking region, wherein the first blocking domain of the second guide strand and the blocking domain of the first guide strand together form a blocking domain that blocks strand displacement activity of a polymerase, the first junction domain of the second guide strand comprises a nucleotide sequence substantially complementary to a nucleic acid sequence of the junction domain of the first guide strand to form a double-stranded region, and the first synthesis region of the second guide strand comprises a nucleotide sequence substantially identical to a nucleic acid sequence of the junction domain of the first guide strand;
(d) optionally, cross-linking the first blocking domain of the second guide strand and the blocking domain of the first guide strand;
(e) further extending the nucleotide sequence;
(f) optionally, adding additional new guide strands to the synthesis reaction and further extending the nucleotide sequence prior to addition of each additional new guide strand, wherein each additional guide strand comprises in a 3′ to 5′ direction a blocking region, a junction domain, a first synthesis region, a second synthesis region, a second junction domain, and a second blocking region, wherein the first blocking region of the new guide stand and the second blocking region of the last guide strand added to the synthesis reaction together form a blocking domain that blocks strand displacement activity of a polymerase; the first junction domain of the new guide strand comprises a nucleotide sequence substantially complementary to a nucleic acid sequence of the second junction domain of the last guide strand added to the synthesis reaction to form a double-stranded region; and the first synthesis region of the new guide strand comprises a nucleotide sequence substantially identical to a nucleic acid sequence of the second junction domain of the last guide strand added to the synthesis reaction;
(g) optionally, cross-linking the first blocking region of the new guide stand and the second blocking region of the last guide strand added to the synthesis reaction prior to further extending the nucleotide sequence.

46) The method of claim 45, further comprising:
(h) adding a terminal guide strand to synthesis reaction, where the terminal guide strand comprises in a 3′ to 5′ direction a blocking region, a junction domain, a first synthesis region, a second synthesis region, a second junction domain, and a second blocking region, wherein the first blocking region of the terminal guide stand and the second blocking region of the last guide strand added to the synthesis reaction together form a blocking domain that blocks strand displacement activity of a polymerase; the first junction domain of the terminal guide strand comprises a nucleotide sequence substantially complementary to a nucleic acid sequence of the second junction domain of the last guide strand added to the synthesis reaction to form a double-stranded region; and the first synthesis region of the terminal guide strand comprises a nucleotide sequence substantially identical to a nucleic acid sequence of the second junction domain of the last guide strand added to the synthesis reaction;
(i) optionally, cross-linking the first blocking domain of the terminal guide strand and the blocking domain of the last guide strand added to the reaction; and
(j) further extending the nucleotide sequence. 47) The method of claim 45 or 46, further comprising amplifying the nucleic acid sequence

48) Use of a nucleic acid template of any one of paragraphs 1-28 for creating combinatorial barcodes to assign unique identities to a target population, wherein the target population is surface positions/partitions, matrices, biomolecules, molecular libraries or biological material (such as vesicles, cells, tissues, organoids, droplets, liposomes, small molecules, beads), and optionally the use comprising splitting the target population prior to addition of a guide strand and pooling the split target population after addition of the guide strand.

49) Use of a method of any one of paragraphs 29-30 for creating combinatorial barcodes to assign unique identities to a target population, wherein the target population is surface positions/partitions, matrices, biomolecules, molecular libraries or biological material (such as vesicles, cells, tissues, organoids, droplets, liposomes, small molecules, beads), and optionally the use comprising splitting the target population prior to addition of a guide strand and pooling the split target population after addition of the guide strand.

50) Use of a reaction mixture of any one of paragraphs 31-42 for creating combinatorial barcodes to assign unique identities to a target population, and wherein the target population is surface positions/partitions, matrices, biomolecules, molecular libraries or biological material (such as vesicles, cells, tissues, organoids, droplets, liposomes, small molecules, beads), and optionally the use comprising splitting the target population prior to addition of a guide strand and pooling the split target population after addition of the guide strand.

51) Use of a method of any one of paragraphs 42-47 for creating combinatorial barcodes to assign unique identities to a target population, wherein the target population is surface positions/partitions, matrices, biomolecules, molecular libraries or biological material (such as vesicles, cells, tissues, organoids, droplets, liposomes, small molecules, beads), and optionally the use comprising splitting the target population prior to addition of a guide strand and pooling the split target population after addition of the guide strand.

52) Use of a nucleic acid template of any one of paragraphs 1-28 for creating combinatorial barcodes to assign spatial position identifiers to a target, wherein the target is a surface, biomolecule, or biological material (including but not limited to biomolecules, cells, tissues, organs, organoids, vesicles, liposomes, and droplets).

53) Use of a method of any one of paragraphs 29-30 for creating combinatorial barcodes to assign spatial position identifiers to a target, wherein the target is a surface, biomolecule, or biological material (including but not limited to biomolecules, cells, tissues, organs, organoids, vesicles, liposomes, and droplets).

54) Use of a reaction mixture of any one of paragraphs 31-37 for creating combinatorial barcodes to assign spatial position identifiers to a target, wherein the target is a surface, biomolecule, or biological material (including but not limited to biomolecules, cells, tissues, organs, organoids, vesicles, liposomes, and droplets).

55)Use of a kit of any one of paragraphs 38-42 for creating combinatorial barcodes to assign spatial position identifiers to a target, wherein the target is a surface, biomolecule, or biological material (including but not limited to biomolecules, cells, tissues, organs, organoids, vesicles, liposomes, and droplets).

56) Use of a method of any one of paragraphs 43-47 for creating combinatorial barcodes to assign spatial position identifiers to a target, wherein the target is a surface, biomolecule, or biological material (including but not limited to biomolecules, cells, tissues, organs, organoids, vesicles, liposomes, and droplets).

It should be understood that this disclosure is not limited to the particular methodology, protocols, and reagents, etc., provided herein and as such may vary. The terminology used herein is for the purpose of describing particular embodiments only, and is not intended to limit the scope of the present disclosure, which is defined solely by the claims. The invention is further illustrated by the following example, which should not be construed as further limiting.

### EXAMPLES

### Example 1

### A Method for Synthesizing Strands Across Nucleic Acid Junctions

The ability to synthesize arbitrary sequences of nucleic acids, in particular DNA, has revolutionized how scientists can study and engineer biology. Whole genomes can now be sequenced effectively (Schwarze et al., 2018) but the technology to synthesize genomes has lagged behind. Synthetic single-stranded sequences (‘oligos’) are typically synthesized chemically through cyclic coupling steps (Caruthers et al., 1987). However, oligos are typically not chemically synthesized past two hundred bases, due to the limitations on chemical coupling efficiency. Therefore, synthesis methods have been developed to assemble larger fragments of single-stranded and double-stranded sequences, including enzymatic assembly using sequential or simultaneous combination of multiple enzyme activities (such as restriction enzyme digestion and ligation, or isothermal Gibson assembly (Gibson, 2011; Gibson et al., 2009) which combines 5′ exonuclease, the 3′ extension activity of a DNA polymerase and DNA ligase activity), non-enzymatic twin primer assembly (Liang et al., 2017), and chemical assembly (such as click DNA assembly (Kukwikila et al., 2017)). While these can be effective at producing longer sequences of single-stranded and double-stranded DNA, sequences of hundreds of thousands of bases must be further assembled from shorter fragments in vivo using yeast vectors (Hutchison et al., 2016). A new simple and robust synthesis method capable of generating long sequences of DNA could have important enabling applications in molecular biology, genome engineering, nanotechnology, and polymer-based data storage. A method for synthesizing strands of nucleic acid across different nucleic acid backbones hybridized together using a strand displacing polymerase is provided herein. This method for synthesizing strands across nucleic acid junctions can effectively decouple the assembly step of template oligos from the synthesis step. In this way it is possible to sequentially perform robust annealing of template oligos, that can hybridize in predictable ways close to thermodynamic optimum, and follow up with polymerization to create the new transcript. This method has the potential to scale to very long and arbitrary sequences. Moreover, the entire assembly and synthesis process can happen isothermally, including at room temperature, which enables a generalized workflow for synthesis of long arbitrary sequences under relatively mild conditions.

The fundamental strategy for cross-junction synthesis is depicted in FIG. 1A-1B. Shown are two template oligos that have been hybridized together to form a junction, and a primer that has bound in front of that junction on the first template strand (left). A strand displacing polymerase is used to copy the x domain until it reaches a stopper (shown in black). Afterwards, the new and old x domains compete in a random walk branch migration process (Lee et al., 1970). Ultimately, the new x domain can bind to the exposed x* domain on the second template strand (right), thus successfully crossing the junction. Polymerization can then continue on the second template strand, copying along a new backbone.

By hybridizing multiple template oligos together, cross junction synthesis reactions can be cascaded to form longer sequences, as depicted in FIG. 2. Each junction between template strands shows the same domain motifs, whereby the strand domains copied on the 3′ end of the growing strand before reaching a stopper (b, c, d, e) can reach across the junction and bind to the exposed complementary sequence (b*, c*, d*, e*) on the next template strand. Arbitrary sequences can be added in the template regions between the motif domains (shown in gray) to enable longer sequences to be assembled.

This method is able to concatenate arbitrary prescribed sequences, and only requires the fundamental domain constraints depicted in FIG. 1A-1B.

It is also suitable for synthesizing specific numbers of repeated sequences through the use of programmable unique hybridization domains that fix the length of the template concatemer, which has been a long-standing challenge. By harnessing the specificity of DNA self-assembly, unique binding sequences promote assemblies of prescribed length, even if the domains being synthesized are identical (FIG. 3).

A strategy for assembling cross-junctions with light will utilize a photoreactive inter-strand crosslinking base, which can serve the dual purpose of linking two cross-junction strands together and acting as a stopper for polymerase synthesis (FIG. 4A-4C). Cross-junctions can be assembled iteratively through cycles of hybridization, crosslinking and washing. Cross-junctions can also be assembled and spatially addressed on a surface, and subsequent synthesis steps can still be performed on the surface.

Assembling a library of orthogonal cross-junction arrays on a surface can enable multiplexed synthesis of orthogonal, and arbitrary length DNA of prescribed sequences.

In the current implementation, a 3-Cyanovinylcarbazole (CNVK) (Vieregg et al., 2013) base modification and a UV light source to crosslink DNA junctions together was used. Strand design can utilize a set of two or more hybridization domains (1 and 1*, 2 and 2* in current implementation), an internal barcode sequence (b 1, b2, etc. . . . ) that will serve as the growing sequence chain, and the CNVK base crosslinker (FIG. 4A, cyan circle).

### Experimental Validation

Cascaded cross junction synthesis, as well as the stepwise assembly of template concatemers on a surface using photoreactive crosslinking chemistry (see above) followed by cross junction synthesis, were validated experimentally.

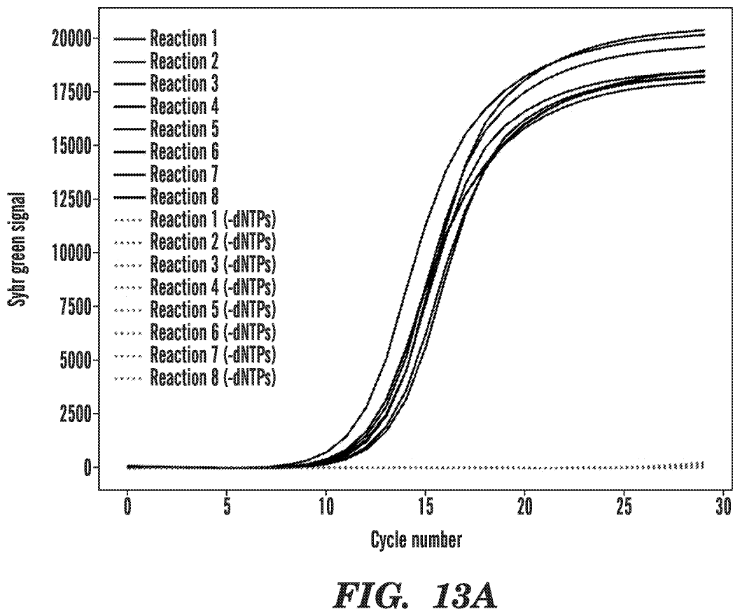

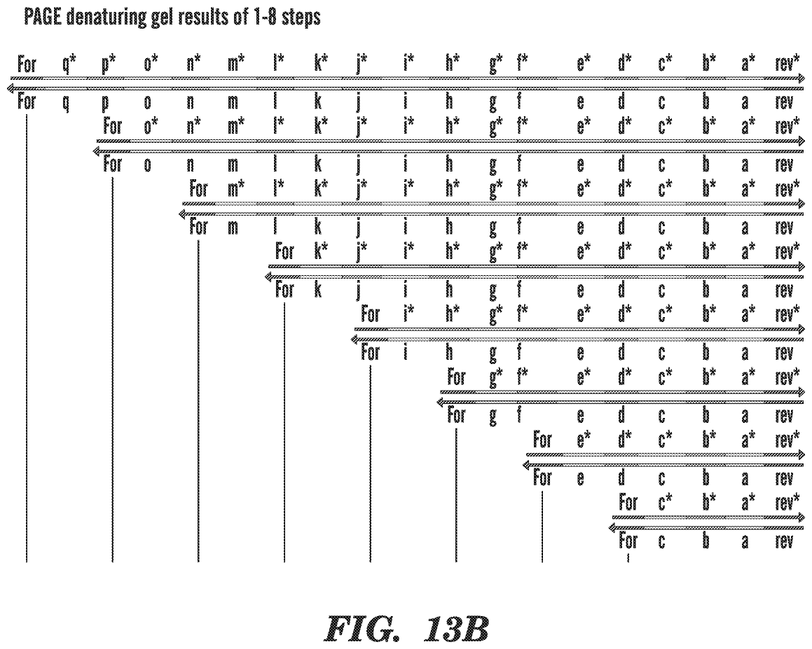

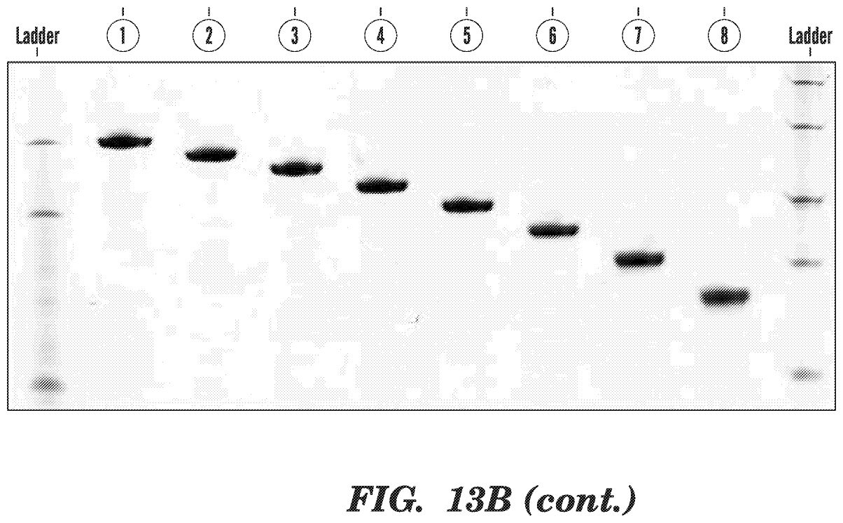

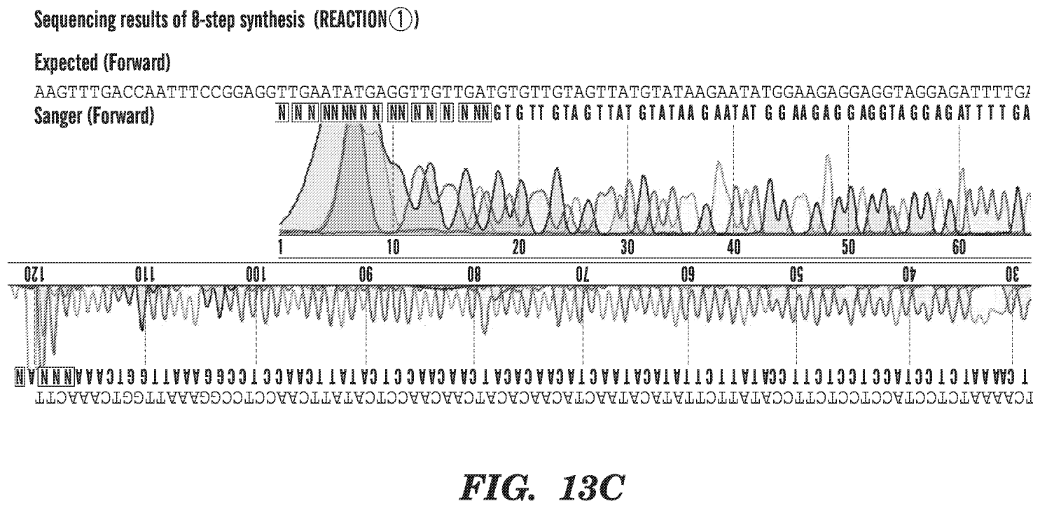

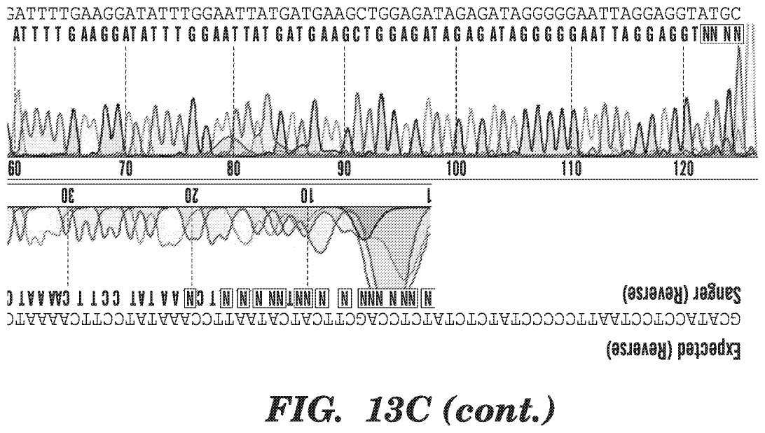

Synthesis cascades ranging from one to eight-junction synthesis as depicted in FIGS. 5-12 were designed utilizing random three-letter (A, T, and C) 7nt ‘flap’ primers (sequences b, d, f, h, j, l, and n), three consecutive G's as a stopper (with dGTP excluded from dNTP mix), random four-letter 9nt secondary hybridization domains (r0, r1, . . . , r7), and 5nt three-letter (A, T, and C) intercalating domains (c, e, g, i, k, m, o, q). Template concatemers were formed by combining strands together at 1 uM concentration in 1× PBS and annealing from 80 C to 20 C over one hour (reducing one degree per minute). Concatemers were then introduced to the synthesis reaction at a final concentration of 50 nM in 0.95×PBS with 10 mM MgSO₄ with the a primer at 40 nM, a dGTP cleanup hairpin at 1 uM, 600 uM dATP/dCTP/dTTP mix, and 800 units/ml of Bst Large Fragment polymerase. Reactions were incubated at room temperature for 15 minutes before the primer was added and incubation continued for a further 30 minutes. 2 uL of 6 mM dGTP was added to 20 uL reactions and incubated for a further 5 minutes to complete the copying of the For primer sequence. Reactions were then heat inactivated for 20 minutes at 80 C before being diluted 1000× (final) into a Taq 1× PCR with 200 nM For and rev primers. A qPCR machine was used to track amplification in real-time using the Sybr Green I intercalating time (FIG. 13A), and PCR products were run on a PAGE denaturing gel (FIG. 13B). The longest, eight-junction synthesis product was further validated using Sanger sequencing (FIG. 13C).

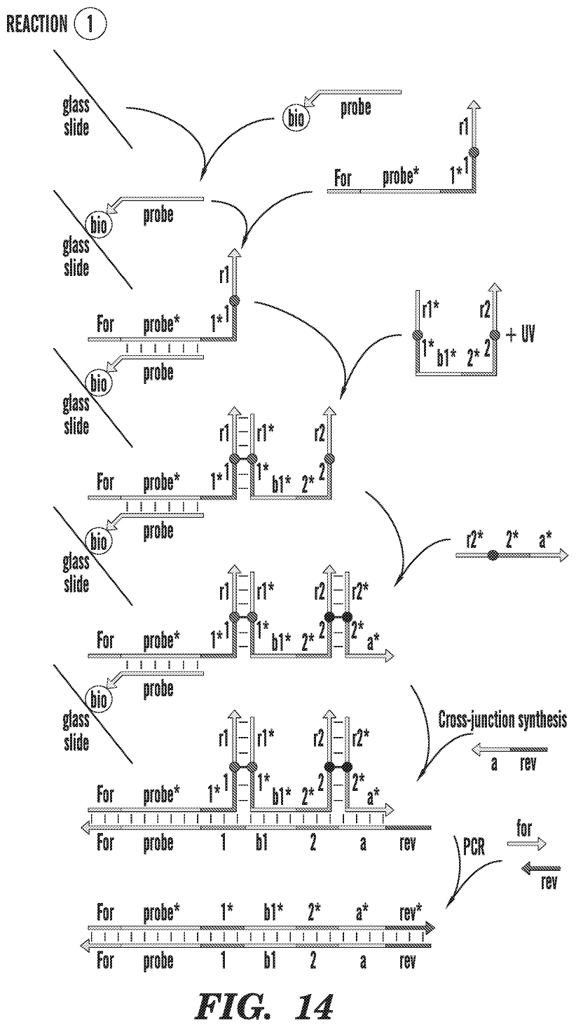

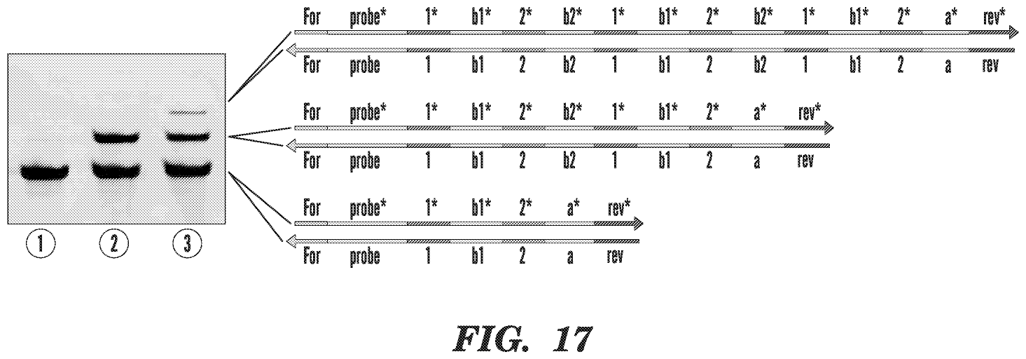

The light-directed concatemer formation (see above) was also experimentally validated. The reactions performed are depicted in FIGS. 14-16, and the result is shown in FIG. 17. After probe sequences were bound to glass slides, template strands were hybridized at 1 uM concentration in 1× PBS for 5 minutes, bound strands were crosslinked with UV light for ˜3 seconds, and then several wash steps were performed before the next hybridization step with 1× PBS to remove unbound strands. All of these steps took place at room temperature. After template formation, cross junction synthesis was performed with 40 nM primer at room temperature, and the result was heat inactivated at 80C for 20 minutes before being diluted into a PCR solution and amplified to form the double stranded product. A PAGE denaturing gel was run to show the products for three different template sizes (FIG. 17).

### Discussion

The novel ability to program synthesis across nucleic acid junctions enables a new paradigm of synthesis that is no longer limited to copying along a single backbone. The formation of the template concatemer (the assembly step) can be performed under favorable conditions, such as through annealing that can help to improve the specificity of the formed hybridization interactions. The primer strand may be included in the formation step or hybridized later, and cross junction synthesis proceeds with a strand displacing polymerase to produce sequence records of the template concatemers. As shown above, arbitrary programmable sequences can be assembled in this way to produce long single-stranded sequences. Single-stranded sequences can either directly be used or amplified (such as with PCR) to form single-stranded or double-stranded products. This capability opens up a number of exciting applications ranging from the generation of combinatorial libraries of sequences to in situ synthesis.

### References

1. Caruthers, M. H., Barone, A. D., Beaucage, S. L., Dodds, D. R., Fisher, E. F., McBride, L. J., Matteucci, M., Stabinsky, Z., and Tang, J.-Y. (1987). [15]Chemical synthesis of deoxyoligonucleotides by the phosphoramidite method. In Methods in Enzymology, (Academic Press), pp. 287-313.
2. Gibson, D. G. (2011). Chapter fifteen-Enzymatic Assembly of Overlapping DNA Fragments. In Methods in Enzymology, C. Voigt, ed. (Academic Press), pp. 349-361.
3. Gibson, D. G., Young, L., Chuang, R.-Y., Venter, J. C., Hutchison, C. A., 3rd, and Smith, H. O. (2009). Enzymatic assembly of DNA molecules up to several hundred kilobases. Nat. Methods 6,343-345.
4. Hutchison, C. A., 3rd, Chuang, R.-Y., Noskov, V. N., Assad-Garcia, N., Deerinck, T. J., Ellisman, M. H., Gill, J., Kannan, K., Karas, B. J., Ma, L., et al. (2016). Design and synthesis of a minimal bacterial genome. Science 351, aad6253.
5. Kukwikila, M., Gale, N., El-Sagheer, A. H., Brown, T., and Tavassoli, A. (2017). Assembly of a biocompatible triazole-linked gene by one-pot click-DNA ligation. Nat. Chem. 9,1089-1098.
6. Lee, C. S., Davis, R. W., and Davidson, N. (1970). A physical study by electron microscopy of the terminally repetitious, circularly permuted DNA from the coliphage particles of Escherichia coli 15. J. Mol. Biol. 48,1-22.
7. Liang, J., Liu, Z., Low, X. Z., Ang, E. L., and Zhao, H. (2017). Twin-primer non-enzymatic DNA assembly: an efficient and accurate multi-part DNA assembly method. Nucleic Acids Res. 45, e94.
8. Schwarze, K., Buchanan, J., Taylor, J. C., and Wordsworth, S. (2018). Are whole-exome and whole-genome sequencing approaches cost-effective? A systematic review of the literature. Genet. Med. 20,1122-1130.
9. Vieregg, J. R., Nelson, H. M., Stoltz, B. M., and Pierce, N. A. (2013). Selective nucleic acid capture with shielded covalent probes. J. Am. Chem. Soc. 13, 9691-9699.

### Example 2

### Nucleic Acid Synthesis Across Multiple Junctions

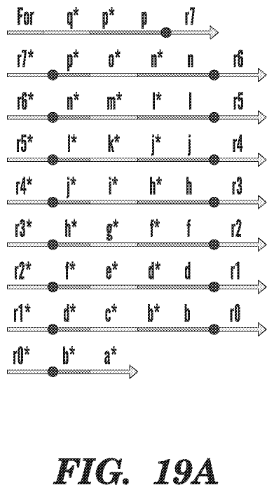

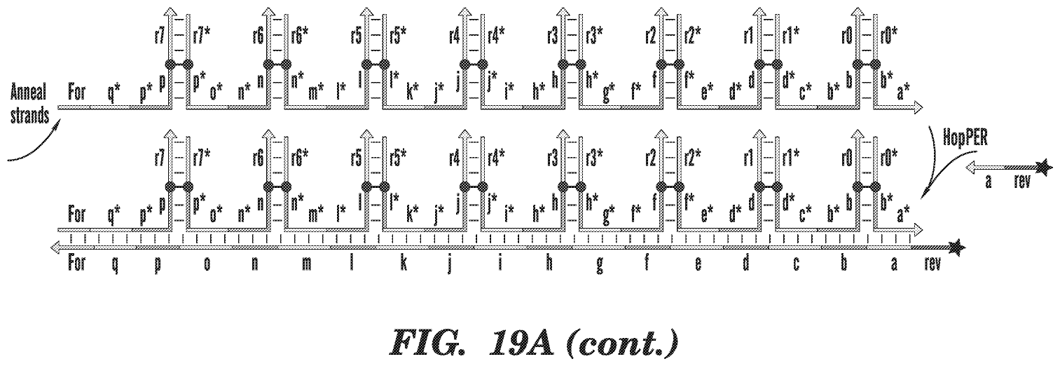

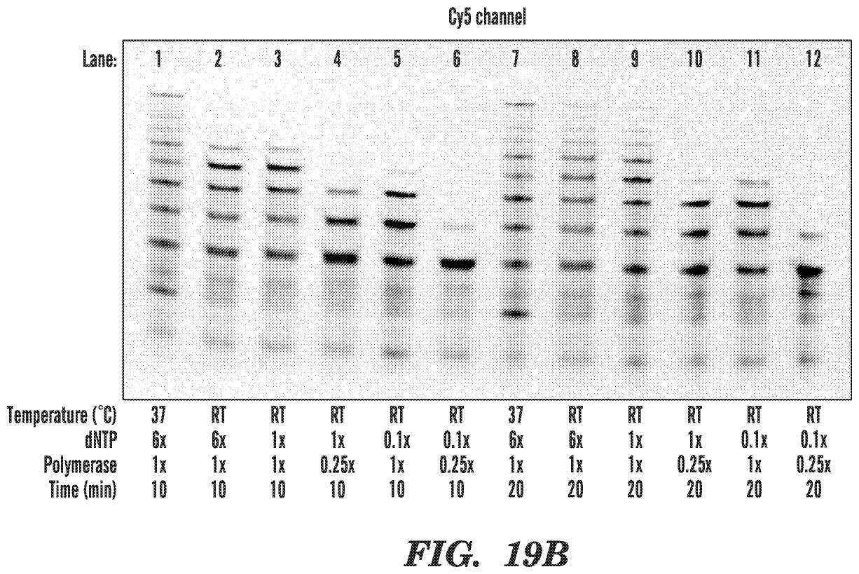

In addition to the methods described above, synthesis across 8-junctions was performed using a fluorophore-labeled primer following the process outlined in FIG. 19A. Template strands (also called guide strands) were annealed (1 μM each strand in 1×PBS, from 80° C. to 20° C. over 1 hour) and diluted into various reaction buffer conditions at two time points (10 and 20 minutes). The template sequences utilized G-C pairs to stop polymerization, and reactions were pre-incubated for 5 minutes with a hairpin. The hairpin can sequester any extraneous dGTP in a mix of dATP, dTTP, and dCTP before the fluorophore-labeled primer can be added (at 40 nM final, with each template strand at 100 nM). Reactions were then incubated for two minutes at the depicted temperature for 10 or 20 minutes before dGTP was added. This step was followed by heat inactivation at 80° C. for 20 minutes. Reactions were run on a 15% TBE-Urea PAGE denaturing gel to determine the effect on synthesis efficiency (FIG. 19B). The two time points (10 and 20 minutes) allowed for monitoring of the reaction process over time. The more bands that appeared on the gel indicated that synthesis had occurred across a higher number of junctions. Lanes 1 and 7 were found to have the highest efficiency for nucleic acid synthesis using the method shown in FIG. 19A (FIG. 19B).

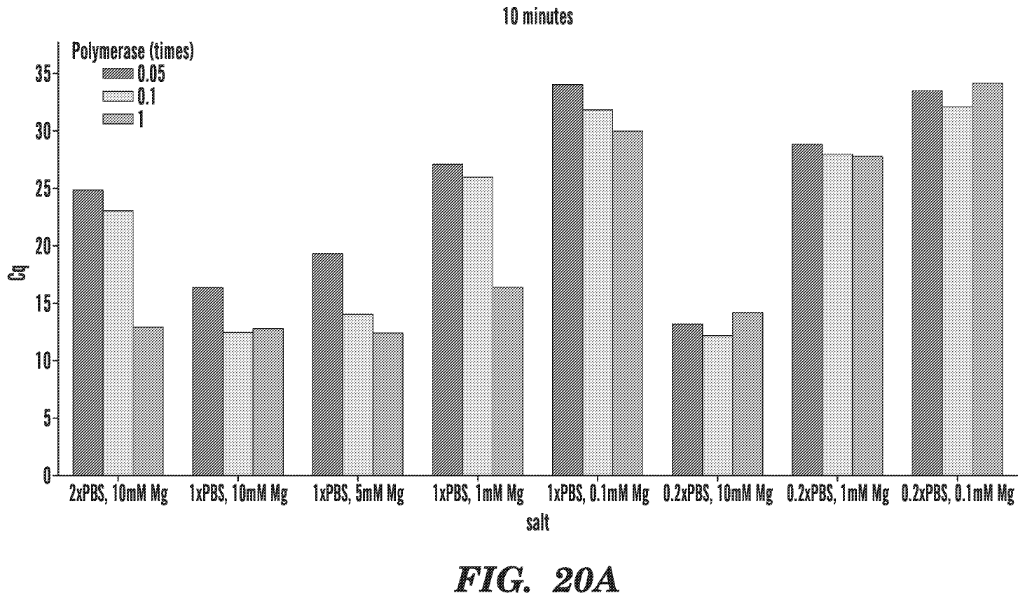

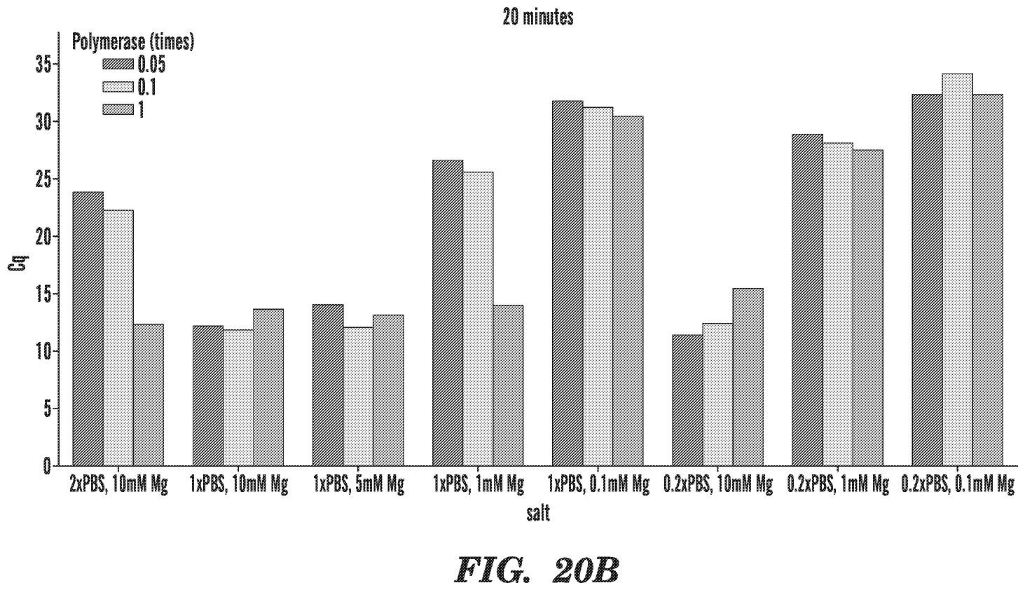

Several salt conditions were also tested with a 5-junction cross-synthesis reaction. In this embodiment of the methods described herein, longer binding domains between template strands were used. The primer and template strands were annealed together at increasing concentrations in 1×PBS, from 80° C. to 20° C. over a 1-hour incubation period. A total of 48 reaction conditions (8 salt conditions×3 polymerase concentrations×two time points) were tested with incubations at 37° C. followed by incubation with dGTP and heat inactivation at 80° C. for 20 minutes. Efficiency was evaluated based on the Cq value from amplification of the full reaction products (FIG. 20A-20B).

In another example, cross junction synthesis was performed by continuously adding one template strand at a time rather than pre-assembly of the template strands as described above (see schematic in FIG. 21A). In continuous nucleic acid synthesis, initially the full reaction conditions for a 5-junction cross-synthesis reaction minus any oligos are pre-incubated before the primer is introduced. After every 2 minutes of incubation at 37° C., a new template strand was added, in the order of synthesis. To ensure nucleic acid synthesis was occurring while each template strand was continuously added, a separate strand (terminal barcode strand) was added. The terminal barcode strand used in this example comprised (i) one or more substantially identical binding domains to the third template strand; and (ii) a different barcode. The barcode is not complementary to the template strand. The terminal barcode strand was added at the end of the synthesis process. Finally, the reaction mixture was incubated with dGTP and was heat inactivated. The complete product was amplified by PCR and Sanger sequencing was performed (FIG. 21B). The sequencing results show the nucleic acid sequences without interference of the barcode from the last terminal barcode strand (FIG. 21B, bottom).

All patents and other publications identified are expressly incorporated herein by reference for the purpose of describing and disclosing, for example, the methodologies described in such publications that might be used in connection with the present disclosure. These publications are provided solely for their disclosure prior to the filing date of the present application. Nothing in this regard should be construed as an admission that the inventors are not entitled to antedate such disclosure by virtue of prior disclosure or for any other reason. All statements as to the date or representation as to the contents of these documents are based on the information available to the applicants and do not constitute any admission as to the correctness of the dates or contents of these documents.

What is claimed is:

1. A nucleic acid template comprising:
(a) a first guide strand comprising in 3′ to 5′ direction a first synthesis region, a second synthesis region, a first junction domain, and a first blocking region; and
(b) a second guide strand comprising in 3′ to 5′ direction a second blocking region, a second junction domain and a third synthesis region,
wherein the first junction domain comprises a nucleotide sequence substantially identical to a nucleotide sequence of the third synthesis region, and the first junction domain and second junction domain are substantially complementary to each other and form a double-stranded region, and
wherein the first blocking region and the second blocking region together form a first blocking domain that blocks strand displacement activity of a polymerase.

2. The nucleic acid template of claim 1, wherein the first and the second blocking regions are covalently linked to each other.

3. The nucleic acid template of claim 1, wherein the first or the second blocking region comprises a cross-linking segment with the other blocking region to form the first blocking domain.

4. The nucleic acid template of claim 3, wherein the cross-linking segment comprises 3-cyanovinylcarbozole

5. The nucleic acid template of claim 1, wherein the second guide strand further comprises a fourth synthesis region at the 5′ end of the third synthesis region.

6. The nucleic acid template of claim 5, wherein the second guide strand further comprises at its 5′ end in 3′ to 5′ direction a third junction domain and a third blocking region, and the nucleic acid template further comprises a third guide strand comprising in 3′ to 5′ direction a fourth blocking region, a fourth junction domain and a fifth synthesis region,
wherein the third junction domain comprises a nucleotide sequence substantially identical to a nucleotide sequence of the fifth synthesis region,
wherein the third junction domain and fourth junction domain are substantially complementary to each other and form a double-stranded region, and
wherein the third blocking region and the fourth blocking region together form a second blocking domain that blocks strand displacement activity of a polymerase.

7. The nucleic acid template of claim 6, wherein the third and the fourth blocking regions are covalently linked to each other.

8. The nucleic acid template of claim 6, wherein the third or the fourth blocking region comprises a cross-linking segment with the other blocking region to form the second blocking domain.

9. (canceled)

10. The nucleic acid template of claim 6, wherein the third guide strand further comprises a sixth synthesis region at the 5′ end of the fifth synthesis region.

11. The nucleic acid template of claim 6, wherein the third guide strand further comprises at its 5′ end in 3′ to 5′ direction a fifth junction domain and a fifth blocking region, and the nucleic acid template further comprises a fourth guide strand comprising in 3′ to 5′ direction a sixth blocking region, a sixth junction domain and a seventh synthesis region,
wherein the fifth junction domain comprises a nucleotide sequence substantially identical to a nucleotide sequence of the seventh synthesis region, and the fourth junction domain and fifth junction domain are substantially complementary to each other and form a double-stranded region, and
wherein the fifth blocking region and the sixth blocking region together form a third blocking domain that blocks strand displacement activity of a polymerase.

12.-14. (canceled)

15. The nucleic acid template of claim 11, wherein the fourth guide strand further comprises an eighth synthesis region at the 5′ end of the sixth synthesis region

16.-19. (canceled)

20. The nucleic acid template of claim 1, wherein one or more regions utilize a 3-letter code.

21. The nucleic acid template of claim 1, wherein at least one of the first, second, third or fourth blocking domain comprises a double stranded region.

22. The nucleic acid template of claim 1, wherein the nucleic acid template comprises a nucleic acid modification.

23. The nucleic acid template of claim 1, wherein at least one of the first, second, third or fourth blocking domain comprises a nucleic acid modification.

24.-25. (canceled)

26. The nucleic acid template of claim 1, wherein the nucleic acid template is conjugated to a solid support.

27. The nucleic acid template of claim 1, wherein the nucleic acid template further comprises a primer annealed or hybridized to the one of the guide strands.

28. (canceled)

29. A method for forming a nucleic acid template, the method comprising annealing or hybridizing a first guide strand and a second guide strand, wherein:
(a) the first guide strand comprises in 3′ to 5′ direction a first synthesis region, a second synthesis region, a first junction domain, and a first blocking region; and
(b) the second guide strand comprises in 3′ to 5′ direction a second blocking region, a second junction domain and a third synthesis region,
wherein the first junction domain comprises a nucleotide sequence substantially identical to a nucleotide sequence of the third synthesis region, and the first junction domain and second junction domain are substantially complementary to each other and form a double-stranded region, and
wherein the first blocking region and the second blocking region together form a blocking domain that blocks strand displacement activity of a polymerase.

30.-42. (canceled)

43. A method for synthesizing a nucleic acid sequence, the method comprising:
(a) providing or obtaining a nucleic acid template of claim 1;
(b) annealing or hybridizing a primer, if not already annealed, to the nucleic acid template; and
(c) extending a nucleotide sequence using a DNA polymerase having strand displacement activity from the 3′-terminus of the primer.

44.-56. (canceled)


SEQUENCE LISTING (6 sequences)

SEQ ID NO: 1 - DNA, 150 bp, Artificial Sequence
  Description of Artificial Sequence Synthetic polynucleotide
aagtttgaccaatttccggaggttgaatatgaggttgttgatgtgttgtagttatgtataagaatatggaagaggaggtaggagattttgaaggatatttggaattatgatgaagctggagatagagatagggggaattaggaggtatgc

SEQ ID NO: 2 - DNA, 125 bp, Artificial Sequence
  Description of Artificial Sequence Synthetic polynucleotide
  modified_base (1)..(17): a, c, t, g, unknown or other
  modified_base (122)..(125): a, c, t, g, unknown or other
nnnnnnnnnnnnnnnnngtgttgtagttatgtataagaatatggaagaggaggtaggagattttgaaggatatttggaattatgatgaagctggagatagagatagggggaattaggaggtnnnn

SEQ ID NO: 3 - DNA, 150 bp, Artificial Sequence
  Description of Artificial Sequence Synthetic polynucleotide
gcatacctcctaattccccctatctctatctccagcttcatcataattccaaatatccttcaaaatctcctacctcctcttccatattcttatacataactacaacacatcaacaacctcatattcaacctccggaaattggtcaaactt

SEQ ID NO: 4 - DNA, 121 bp, Artificial Sequence
  Description of Artificial Sequence Synthetic polynucleotide
  modified_base (1)..(11): a, c, t, g, unknown or other
  modified_base (13)..(17): a, c, t, g, unknown or other
  modified_base (20)..(20): a, c, t, g, unknown or other
  modified_base (117)..(119): a, c, t, g, unknown or other
  modified_base (121)..(121): a, c, t, g, unknown or other
nnnnnnnnnnntnnnnntcnaatatccttcaaaatctcctacctcctcttccatattcttatacataactacaacacatcaacaacctcatattcaacctccggaaattggtcaaannnan

SEQ ID NO: 5 - DNA, 171 bp, Artificial Sequence
  Description of Artificial Sequence Synthetic polynucleotide
atcctagcccatacggcaatgcctatctctatctccagcttcatcaaacccacaccaaataaaccacacaaaatcaaccacacctcctcaaccacatattcttttccagagccttgaattgtaatgctctgtgtcctctatcagacttaagcattcacccttgcacgatac

SEQ ID NO: 6 - DNA, 148 bp, Artificial Sequence
  Description of Artificial Sequence Synthetic polynucleotide
  modified_base (3)..(20): a, c, t, g, unknown or other
  modified_base (25)..(25): a, c, t, g, unknown or other
  modified_base (27)..(27): a, c, t, g, unknown or other
  modified_base (148)..(148): a, c, t, g, unknown or other
ccnnnnnnnnnnnnnnnnnntcaancncaccaaataaaccacacaaaatcaaccacacctcctcaaccacatattcttttccagagccttgaattgtaatgctctgtgtcctctatcagacttaagcattcacccttgcacgatacan